(19) United States
(12) Patent Application Publication
(10) Pub. No.: US 2006/0233785 A1
(43) Pub. Date: Oct. 19, 2006

(54) Immunointeractive molecules and uses thereof

(76) Inventor: Robyn O'Hehir, Parkville (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/494,461

(22) PCT Filed: Oct. 30, 2002
(86) PCT No.: PCT/AU02/01473

§ 371 Date: Dec. 28, 2004

(30) Foreign Application Priority Data
Oct. 30, 2001 (AU) PR 8490

(51) Int. Cl. A61K 38/17; A61K 39/395; G01N 33/53; C07K 14/415; C12P 21/06

(52) U.S. Cl. 424/130.1; 514/12; 514/13; 530/324; 530/326; 435/69.1; 435/320.1; 435/325; 435/326; 530/387.1; 435/7.1

(57) ABSTRACT

The present invention relates generally to molecules such as peptides, polypeptides and proteins which interact immunologically with T lymphocytes in subjects having latex allergy and genetic sequences encoding the same. These molecules are preferentially immunointeractive with T cells in subjects having a Hev b 6 allergy. The present invention also extends to antibodies, preferably monoclonal antibodies, directed to latex allergens and in particular to Hev b 6, and to the B cell epitopes recognized therein. The molecules of the present invention are useful in the development of diagnostic, therapeutic and prophylactic agents for conditions characterised by an aberrant, inappropriate or otherwise unwanted immune response to Hev b 6 or derivatives or homologues thereof.

### FIELD OF THE INVENTION

The present invention relates generally to molecules such as peptides, polypeptides and proteins which interact immunologically with T lymphocytes in subjects having latex allergy and genetic sequences encoding same. These molecules are preferentially immunointeractive with T cells in subjects having a Hev b 6 allergy. The present invention also extends to antibodies, preferably monoclonal antibodies, directed to latex allergens and in particular to Hev b 6, and to the B cell epitopes recognised therein. The molecules of the present invention are useful in the development of diagnostic, therapeutic and prophylactic agents for conditions characterised by an aberrant, inappropriate or otherwise unwanted immune response to Hev b 6 or derivative or homologue thereof.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

IgE mediated hypersensitivity to latex has emerged as a serious occupational health problem since the introduction of universal precautions in the mid 1980s (Slater J E et al., 1996; J Biol Chem., 271:25349-24399; Sussman G L et al., 1991; JAMA., 265:2844-2847). The use of latex gloves for barrier protection may lead to sensitisation, especially of health care workers (HCW), to protein allergens present in the natural rubber latex. The prevalence of latex sensitisation amongst HCW has been reported from 8.2-22% (Grzybowski M et al., 1996; J. Allergy Clin Immunol., 98:535-544; Kibby T and Akl M., 1997; Ann Allergy Asthma Immunol., 78:41-44; Liss G M et al., 1997; Occ Environ Med., 54:335-342; Brown R H et al., 1998; Anaesthesiology., 89:292-299; Douglas R et al., 1997; Aust NZ J Med., 27:165-169). Allergic reactions to latex range from urticaria, rhinoconjunctivitis, asthma (Brugnami G et al., 1995; J Allergy Clin Immunol., 96:457-464), and angioedema to severe generalised anaphylaxis in some cases (Ownby D R et al., 1991; AJR., 156:903-908). Since the only form of treatment available at present is allergen avoidance and symptomatic relief, there is an urgent need for the development of specific immunotherapy for this condition. Thus, the immunological characterisation of latex allergens is an important breakthrough in the development of rational curative treatments for latex allergy.

Up to 52% of latex allergy sufferers also exhibit sensitivity to various fruits and vegetables (Blanco C et al., 1994; Ann Allergy., 73(4):309-14). Also, latex allergic individuals have four times the risk of the general population of food allergy. Food allergy amongst latex-sensitive subjects frequently manifests as anaphylaxis (36% in the Blanco study) (Blanco C et al., 1994; Allergy., 49(6):454-9). Therefore, the latex-fruit syndrome is of considerable clinical importance. More than 20 foods, fruits or plants have been reported to be cross-reactive with latex (Blanco et al., 1994, supra; Alenius H et al., 1996; Clin Exp Allergy., 26(3):341-8; Blanco et al., 1994, supra; Abeck D et al., 1994; Hautarzt., 45(6):364-7; Anliker M D et al., 2001; J Allergy Clin Immunol., 107(4):718-23; Antico A., 1996; Ann Allergy Asthma Immunol., 76(1):37-40; De Greef J M et al., 2001; Int Arch Allergy Immunol., 125(2):182-4; Duque S et al., 1999; Allergy., 54(9):1004-5; Garcia Ortiz J C et al., 1998; Allergy., 53(5):532-6; Levy D A et al., 2000; Clin Exp Allergy., 30(2):270-5; Sanchez-Guerrero I M et al., 2000; Allergy., 55(10):976-7; Seppala U et al., 2000; Allergy., 55(3):266-73 and Weiss S J et al., 1996., Ann Allergy Asthma Immunol., 77(6):504-8). The most commonly described are banana, avocado, kiwifruit and chestnut. Table 1 however, shows the fruits and vegetables reported cover a wide proportion of the plant kingdom. This is indicative of important issues: firstly, that a pan-allergen (or allergens) is likely important, and secondly, it shows the breadth of dietary components that may put severely latex-allergic individuals at risk of food allergy symptoms.

It is not currently advised for latex allergic patients to avoid all such foods unless they have specific problems. However, it is incumbent upon clinicians to warn patients of the potential for these reactions.

Several allergens from the rubber plant Hevea brasiliensis have been identified (Chen Z et al., 1996; Clin Exp Allergy., 26:406-415; Yeang H. Y. et al., 1996., J Allergy Clin Immunol; 98:628-639; Sowka S et al., 1998; Eur J Bioch., 255:213-219; Scheiner O et al., 1999; Int Arch Allergy Immunol., 118:311-312; Kostyal D A et al., 1998; Clin Exp Immunol., 112:355-362; Akasawa A et al., 1996; J Biol. Chem., 271:25389-25393; Slater et al., 1996, supra; Chen Z et al., 1998; J Allergy Clin Immunol., 102:476-481; Posch A et al., 1998; Clin Exp Allergy., 28:134-140). The major latex allergen Hev b 6 has been identified as being the principal latex allergen responsible for the cross-reactivity discussed above (Mikkola J H et al., 1998; J Allergy Clin Immunol., 102(6 Pt 1): 1005-12). The hevein molecule (Hev b 6.02) has striking homology with Class 1 chitinases (Blanco C et al., 1994; Allergy., 49(6):454-9) which have a hevein-like domain and occur widely in plants, thus fitting the description of a “pan-allergen”. RAST inhibition and skin testing studies have provided clinical correlation of this molecular observation. Although primary sensitisation via fruit exposure has been reported and is a risk factor for latex allergy (Garcia et al., 1998, supra), current evidence indicates that primary sensitisation to latex accounts for the vast bulk of the latex-fruit syndrome. Whether other latex allergens are responsible for food cross-reactivity is not so clear.

The production of allergen specific IgE by B cells and release of inflammatory mediators by mast cells and eosinophils result in the effector response of allergic disease. However, it is well established that these events are orchestrated by allergen-specific CD4⁺ T cells with a Th₂-type cytokine profile. T cell reactive determinants have been reported for another major latex allergen, Hev b 1 (Raulf-Heimsoth M et al., 1998; Clin Exp Allergy., 28:339-348), but not for Hev b 6. Thus, characterisation of the immune response to Hev b 6 is critical in the development of specific diagnostic and immunotherapeutic methodology. In work leading up to the present invention, the inventors have identified the human T cell epitopes of the latex allergen, Hev b 6. The inventors have also developed monoclonal antibodies directed to Hev b 6 thereby facilitating the identification of Hev b 6 B cell epitopes. The identification of Hev b 6 T and B cell epitopes and the production of monoclonal antibodies to Hev b 6 now facilitates the development of molecules and methodology for the diagnosis and treatment of conditions characterised by the aberrant, inappropriate or otherwise unwanted immune response to Hev b 6 or derivative or homologue thereof such as latex allergy or plant hypersensitivity.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The subject specification contains amino acid and nucleotide sequence information prepared using the programme PatentIn Version 3.1, presented herein after the bibliography. Each amino acid or nucleotide sequence is identified in the sequence listing by the numeric indicator <201> followed by the sequence identifier (eg. <210>1, <210>2, etc). The length, type of sequence (DNA, protein, etc) and source organism for each amino acid or nucleotide sequence is indicated by information provided in the numeric indicator fields <211>, <212> and <213>, respectively. Amino acid and nucleotide sequences referred to in the specification are identified by the indicator SEQ ID NO: followed by the sequence identifier (eg. SEQ ID NO:1, SEQ ID NO:2, etc.). The sequence identifier referred to in the specification correlates to the information provided in numeric indicator field <400> in the sequence listing, which is followed by the sequence identifier (eg. <400>1, <400>2, etc). That is SEQ ID NO:1 as detailed in the specification correlates to the sequence indicated as <400>1 in the sequence listing.

One aspect of the present invention provides an isolated peptide of the formula:
X₁X₂X₃
wherein:
X₁ and X₃ may be the same or different and each is an amino acid sequence comprising from 0 to 40 naturally or non-naturally occurring amino acid residues;
X₂ is any amino acid sequence derived from or homologous to Hev b 6;
   and wherein said peptide molecule is capable of interacting with T cells and modifying T cell function when incubated with cells from subjects having a condition characterised by an aberrant, unwanted or otherwise inappropriate immune response to Hev b 6 or a derivative, homologue, analogue, mutant, chemical equivalent or mimetic of said peptide.

The present invention therefore more particularly provides an isolated peptide of the formula:
X₁X₂X₃
wherein:
X₁ X₃ may be the same or different and each is an amino acid sequence comprising from 0 to 40 naturally or non-naturally occurring amino acid residues;
X₂ is an amino acid sequence of from 5 to 100 residues derived from, homologous to or contiguous with amino acids 1-187 inclusive or derivatives thereof of Hev b 6;
   and wherein said peptide molecule is capable of interacting with T cells and modifying T cell function when incubated with cells from subjects having a condition characterised by an aberrant, unwanted or otherwise inappropriate immune response to Hev b 6 or a derivative, homologue, mutant, chemical equivalent or mimetic of said peptide.

Still more particularly the present invention provides an isolated peptide of the formula:
X₁X₂X₃
wherein
X₁ and X₃ may be the same or different and each is an amino acid sequence comprising from 0 to 40 naturally or non-naturally occurring amino acid residues;
X₂ is an amino acid sequence of from 5 to 100 residues derived from, homologous to or contiguous with amino acids 1-47, 55-83, 82-101 and/or 136-155 inclusive or derivatives thereof of Hev b 6;
   and wherein said peptide molecule is capable of interacting with T cells and modifying T cell function when incubated with cells from subjects having a condition characterised by an aberrant, unwanted or otherwise inappropriate immune response to Hev b 6 or a derivative, homologue, analogue, mutant, chemical equivalent or mimetic of said peptide.

Still more particularly, X₂ is any amino acid sequence of from 5 to 100 residues derived from, homologous to or contiguous with amino acids 1-20, 10-29, 19-38, 28-47, 55-74, 64-83 and/or 136-155 inclusive or derivatives thereof of Hev b 6.

Yet more particularly, X₂ is any amino acid sequence of from 5 to 100 residues derived from homologous to or contiguous with amino acids 1-20, 10-29, 19-38 and/or 2847 inclusive or derivatives thereof of Hev b 6.

Most particularly, X₂ is any amino acid sequence of from 5 to 100 residues derived from, homologous to or contiguous with amino acids 10-29 and/or 19-38 inclusive or derivatives thereof of Hev b 6.

In a particularly preferred embodiment, X₂ comprises a sequence of at least 5 amino acids derived from one or more of the following amino acid sequences:

| | |
|---|---|
| EQCGRQAGGKLCPNNLCCSQ | (SEQ ID NO:2) |
| KLCPNNLCCSQWGWCGSTDE | (SEQ ID NO:3) |
| SQWGWCGSTDEYCSPDHNCQ | (SEQ ID NO:4) |
| DEYCSPDHNCQSNCKDSGEG | (SEQ ID NO:5) |

More preferably, X₂ comprises a sequence of at least 5 amino acids derived from one or more of SEQ ID NO:3 or SEQ ID NO:4.

Another aspect of the present invention provides an isolated peptide comprising any amino acid sequence derived from or homologues to Hev b 6 wherein said peptide molecule is capable of interacting with T cells and modifying T cell function when incubated with cells from subjects having a condition characterised by an aberrant, unwanted or otherwise inappropriate immune response to Hev b 6 or a derivative, homologue, analogue, mutant, chemical equivalent or mimetic of said peptide.

Yet another aspect of the present invention provides an isolated peptide comprising an amino acid sequence of from 5-100 residues derived from, homologues to or contiguous with amino acids 1-187 inclusive or derivatives thereof of Hev b 6 wherein said peptide molecule is capable of interacting with T cells and modifying T cell function when incubated with cells from subjects having a condition characterised by an aberrant, unwanted or otherwise inappropriate immune response to Hev b 6 or a derivative, homologue, analogue, mutant, chemical equivalent or mimetic of said peptide.

In one preferred embodiment said amino acid sequence is derived from, homologous to or contiguous with amino acids 1-47, 55-83, 82-101 and/or 136-155 inclusive or derivatives thereof of Hev b 6.

In another preferred embodiment said amino acid sequence is derived from, homologous to or contiguous with amino acids 1-20, 10-29, 19-38, 28-47, 64-83 and/or 136-155 inclusive or derivatives thereof of Hev b 6.

In yet another preferred embodiment said amino acid sequence is derived from, homologous to or contiguous with amino acids 1-20, 10-29, 19-38 and/or 28-47 inclusive or derivatives thereof of Hev b 6.

More preferably, said amino acid sequence is derived from, homologous to or contiguous with amino acids 10-29 and/or 19-38, inclusive or derivatives thereof of Hev b 6.

In another aspect said amino acid sequence comprises a sequence of at least 5 amino acids derived from one or more of the following amino acid sequences:

| | |
|---|---|
| EQCGRQAGGKLCPNNLCCSQ | (SEQ ID NO:2) |
| KLCPNNLCCSQWGWCGSTDE | (SEQ ID NO:3) |
| SQWGWCGSTDEYCSPDHNCQ | (SEQ ID NO:4) |
| DEYCSPDHNCQSNCKDSGEG | (SEQ ID NO:5) |

According to this aspect, said amino acid sequence preferably comprises a sequence of at least 5 amino acids derived from one or more of SEQ ID NO:3 or SEQ ID NO:4.

Another aspect of the present invention is directed to antibodies to Hev b 6 including catalytic antibodies or derivatives, homologues, analogues, mutants, chemical equivalents or mimetics of said antibodies.

In a preferred embodiment the subject antibody is 1A5.4 or 6E5.3 or derivative, homologue, analogue, chemical equivalent, mutant or mimetic thereof.

In another aspect there is provided an isolated peptide of the formula:
X₁X₂X₃
wherein
X₁ and X₃ may be the same or different and each is an amino acid sequence comprising from 0 to 40 naturally or non-naturally occurring amino acid residues;
X₂ is an amino acid sequence derived from, or homologous to Hev b 6
   and wherein said peptide molecule is capable of interacting with antibody from subjects having a condition characterised by an aberrant, unwanted or otherwise inappropriate immune response to Hev b 6 or a derivative, homologue, analogue, mutant, chemical equivalent or mimetic of said peptide.

Preferably, said condition is latex hypersensitivity and said antibodies are of the IgE isotype.

More particularly the present invention provides an isolated peptide of the formula:
X₁X₂X₃
wherein
X₁ and X₃ may be the same or different and each is an amino acid sequence comprising from 0 to 40 naturally or non-naturally occurring amino acid residues;
X₂ is any amino acid sequence derived from, or homologous to Hev b 6
   and wherein said peptide molecule is capable of interacting with antibodies expressed by hybridomas 1A5.4 or 6E5.3 or a derivative, homologue, analogue, mutant, chemical equivalent or mimetic of said peptide.

In another aspect, the present invention provides a nucleic acid molecule comprising a sequence of nucleotides encoding or complementary to a sequence encoding the peptides as hereinbefore defined or a derivative, homologue or analogue thereof.

In yet another aspect the present invention provides a method for the treatment and/or prophylaxis of a condition in the subject, which condition is characterised by the aberrant, unwanted or otherwise inappropriate immune response to Hev b 6, said method comprising administering to said subject an effective amount of a peptide and/or antibody as hereinbefore defined for a time and under conditions sufficient to remove or reduce the presence or function in said subject of T cells and/or antibodies directed to said Hev b 6.

Still another aspect of the present invention contemplates the use of an agent as hereinbefore defined in the manufacture of a medicament for the treatment of a condition in a mammal, which condition is characterised by an aberrant, unwanted or otherwise inappropriate immune response to Hev b 6.

In yet another further aspect, the present invention contemplates a pharmaceutical composition comprising an agent as hereinbefore defined and one or more pharmaceutically acceptable carriers and/or diluents.

Yet another aspect of the present invention relates to agents, as hereinbefore defined, when used in the method of the present invention.

Still yet another aspect of the present invention is directed to a method of diagnosing or monitoring a condition in a mammal, which condition is characterised by an aberrant, unwanted or inappropriate response to Hev b 6, said method comprising screening for Hev b 6 reactive T cells and/or antibodies utilising a peptide as hereinbefore defined.

Still another aspect of the present invention is directed to a method of qualitatively and/or quantitatively detecting Hev b 6, or peptides thereof, in a sample said method comprising screening for said Hev b 6 or peptides thereof utilising an antibody as hereinbefore defined.

In a further aspect the present invention provides diagnostic kits for use in the diagnostic methodology hereinbefore defined.

Single and three letter abbreviations used throughout the specification are defined in Table 2.

TABLE 2
Single and three letter amino acid abbreviations

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any residue | Xaa | X |

### BRIEF DESCRIPTION OF THE DRAWINGS

FIG. 1 is a graphical representation of an ELISA detecting monoclonal antibody binding to Hev b 6 and Hev b 6 peptides ELISA plates were coated with 2 μg/ml Hev b 6 peptides. rHev b 6 and glove extract and blocked with 5% skim milk powder. The plates were then incubated with Hev b 6-specific hybridoma supernatants 1A5.4, 6E5.3 and 5C3 and Hev b 5-specific monoclonal antibody 6F6 (isotype control for 1A5.4 and 6E5.3). Antibody binding was detected by incubation with HRP-labeled sheep anti-mouse immunoglobulin and o-phenylenediamine as substrate. Mean values+SD for triplicate wells are shown.

FIG. 2 is an image of the immunoblotting of latex allergen preparations with anti-Hev b 6 monoclonal antibody 1A5.4.3. Latex allergen preparations were subjected to electrophoresis under reducing conditions on a 16% SDS PAGE minigel and then either stained with Coomassie Brilliant Blue (A) or transferred to a nitrocellulose membrane (B). For immunoblotting, the membrane was blocked with 5% skim milk powder, and then incubated with 1A5.4.3 hybridoma supernatant, followed by HRP-labeled sheep anti-mouse immunoglobulin and 4-chloro-1-naphthol as substrate. M: molecular mass markers; lane 1: glove extract; lane 2: low ammoniated latex; lane 3: non-ammoniated latex; lane 4: rHev b 6; lane 5: herein.

FIG. 3 is a graphical representation of an ELISA detecting latex-specific serum IgE. ELISA plates were coated with 10 μg/ml rHev b 6 and glove extract, blocked with skim milk powder and incubated with patient (P1-10) and non-allergic control serum (NA1-6) diluted 1:10. Antibody binding was detected by incubation with rabbit anti-human IgE, swine anti-rabbit immunoglobulines-HRP and o-phenylenediamine as substrate. Mean values+SD for triplicate wells are shown. The cut-off for positive was 2 SD above the mean OD of non-allergic subjects for rHev b 6 (dotted line) and glove extract (black line).

FIG. 4 is an image of immunoblotting detecting rHev b 6-specific serum IgE. rHev b 6 was subjected to electrophoresis under reducing conditions on a 16% SDS PAGE minigel and transferred to a nitrocellulose membrane. After blocking with skim milk powder, the membrane was incubated with serum from latex allergic and non-allergic subjects (1:5 dilution). Antibody binding was detected by incubation with rabbit anti-human IgE, swine anti-rabbit immunoglobulins-HRP and 4-chloro-1-naphthol as substrate. M: molecular mass markers; C: Coomassie-stained gel; lane 1: 1A5.4.3 monoclonal antibody; lane 2: P1 serum; lane 3: P4 serum; lane 4: P5 serum; lane 5: non-latex-allergic serum; lane 6: no serum.

FIG. 5 is a graphical representation of a T cell proliferation assay on latex-specific T-cell line from latex-allergic patient P1. Proliferation of latex-specific T-cell line from P1 in response to Hev b 6 peptides (10 μg/ml), glove extract (3-100 μg/ml), sulphonated rHev b 6 (3-100 μg/ml) and IL-2 (10 IU/ml) assessed by ³H-thymidine incorporation (mean cpm+SD for triplicate cultures are shown). Background levels of cell proliferation are shown for T cells with antigen presenting cells (T+APC) alone, T cells alone and APC alone.

FIG. 6 is an image of sulphonation abolishing the IgE reactivity of rHev b 6. rHev b 6 (A) and sulphonated rHev b 6 (B) were resolved by 16% SDS-PAGE under non-reducing conditions. Gels were either stained with Coomassie Brilliant Blue (1) or transferred to nitrocellulose. Strips with transferred protein were incubated with the Hev b 6-specific mAb 1A5.4 and binding detected (2). Strips with transferred protein were also incubated with sera from two latex allergic, Hev b 6 sensitized subjects (3 and 4) and one non-latex-allergic, atopic subject (5). The presence or absence of IgE binding was then detected. (C) ELISA showing lack of reactivity of sulphonated rHev b 6 with IgE from the sera of six latex allergic, Hev b 6 sensitized subjects. Plates were coated with rHev b 6 and sulphonated rHev b 6 and the presence or absence of IgE-binding detected. As a positive control the Hev b 6-specific mAb 1A5 was used. NB indicates no binding of IgE was detected.

FIG. 7 is a graphical representation of the disruption of the first disulphide bond of Hev b 6 markedly decreasing IgE binding to rHev b6. IgE from the sera of 31 latex allergic and 18 non-latex allergic subjects assayed by ELISA for the ability to bind rHev b 6 (no substitution) and mutant rHev b 6 (cysteine to alanine substitution at amino acid 3). Plates were coated with both rHev b 6 and mutant rHev b 6 and incubated with subjects' sera (A) Population ELISA. Individual dots represent individual readings. The thin horizontal lines indicated the cut-off for positive for IgE binding to mutant rHev b 6 and rHev b 6, calculated as the mean of the readings for non-latex allergic subjects for each antigen plus 2 SD. The thick horizontal lines indicate the mean of each group. (B) Direct comparison of IgE and mAb binding to rHev b 6 and mutant rHev b 6. A1 to 11, latex-allergic subjects. N1, non-latex allergic subject. Dotted line indicates the cut-off for positive for IgE binding for mutant rHev b 6. The solid line indicates the cut-off for positive for IgE binding for rHev b 6.

FIG. 8 is a graphical representation of indicating that mutant rHev b 6 is a poor inhibitor of IgE binding to rHev b 6. Inhibition ELISA were carried out to confirm the lack of IgE reactivity of mutant rHev b 6. Various concentrations of mutant rHev b 6 and rHev b 6, ranging from 0.0016 μg/ml to 125 μg/ml were preincubated with patient sera before the sera were used in ELISA to measure IgE binding to rHev b 6. Incubation of sera with KLH was used as a negative control. Values are presented as percent inhibition±standard deviation. (A) The Hev b 6-specific mAb 1A5.4 was incubated with KLH, mutant rHev b 6 and rHev b 6 and mAb binding to rHev b 6 detected. Sera from three latex-allergic, Hev b 6 sensitised subjects A7 (B), A6 (C) and A8 (D) were incubated with KLH, mutant rHev b 6 and rHev b 6 and IgE binding to rHev b 6 detected. The horizontal line indicates the level below which inhibition was considered to non-specific.

FIG. 9 is a graphical representation of the basophil Activation Test on a latex allergic donor. Whole blood from a latex allergic, Hev b 6 sensitized subject was incubated with latex allergens. The cells were stained with FITC labelled anti-human IgE polyclonal antibodies and a PE labelled monoclonal anti-human CD63 antibody. Cells were analysed by flow cytometry defining basophils on the basis of scatter (A) and high IgE staining (B). The percentage of activated basophils expressing CD63 was determined (C).

FIG. 10 is a graphical representation indicating that mutant rHev b 6 is a poor activator of basophils. The Basophil Activation Test was performed on whole blood from (A) non-latex allergic subject N2; (B) latex-allergic subject A7; (C) latex-allergic subject A8 and (D) latex-allergic subject A10. Antigens: latex glove extract (GE), hevein peptide (hevein), rHev b 6, mutant rHev b 6 (MU rHev b 6), sulphonated rHev b 6, (sul. rHev b 6), rHev b 5, KLH and house dust mite extract (HDM). All allergens were used at 10 μg/ml except for rHev b 6 and mutant rHev b 6 which were used at 0.1, 1 and 10 μg/ml. The cut-off for positive was set at the level of activated basophils after incubation in stimulation buffer alone (no antigen, horizontal line). A plus symbol (+) indicates antigen to which a subject has either a positive clinical history of allergy and/or a positive reading on an allergen-specific IgE ELISA.

FIG. 11 is a graphical representation of Hev b 6-specific T-cell lines proliferating in response to mutant rHev b 6. Retention of T-cell stimulatory activity of mutant rHev b 6 was determined in oligoclonal T cell proliferation assays. Oligoclonal T cells (5×10⁴/well) were stimulated with rHev b 6 or mutant rHev b 6 in the presence of equal numbers of irradiated APC and proliferation as correlated with incorporation of tritiated thymidine determined. Results are shown as mean cpm+SD for triplicate cultures.

Control cultures contained no antigen or 10% IL-2.

FIG. 12 is a schematic representation of the generation of new mutants of rHev b 6.

FIG. 13 is a schematic representation of the amino acid sequences of rHev b 6 mutants.

### DETAILED DESCRIPTION OF THE INVENTION

The present invention is predicated, in part, on the identification of Hev b 6 T and B cell epitopic regions and the development of monoclonal antibodies to Hev b 6. The identification of immunodominant epitopes of Hev b 6 has enabled the improvement of diagnostic methodology and the development of therapeutic and prophylactic compositions and treatment approaches for conditions such as, but not limited to, latex allergy.

In accordance with the present invention, overlapping peptides were synthesised based on the Hev b 6 amino acid sequence disclosed in SEQ ID NO:1. The T cell immunoreactivity of these peptides is identified in accordance with the present invention on the basis of interactivity of peripheral blood cells or T cells obtained from the peripheral blood of subjects with latex hypersensitivity. The identification of Hev b 6 B cell epitopes has been facilitated via the generation of a panel of monoclonal antibodies directed to Hev b 6. The identification and generation of those molecules thereby form the basis for a new range of diagnostic, therapeutic and prophylactic reagents and procedures.

Accordingly, one aspect of the present invention provides an isolated peptide of the formula:
X₁X₂X₃
wherein:
X₁ and X₃ may be the same or different and each is an amino acid sequence comprising from 0 to 40 naturally or non-naturally occurring amino acid residues;
X₂ is any amino acid sequence derived from or homologous to Hev b 6;
   and wherein said peptide molecule is capable of interacting with T cells and modifying T cell function when incubated with cells from subjects having a condition characterised by an aberrant, unwanted or otherwise inappropriate immune response to Hev b 6 or a derivative, homologue, analogue, mutant, chemical equivalent or mimetic of said peptide.

Hev b 6 is a protein which has been identified in the rubber plant Hevea brasiliensis. In this regard, reference to “Hev b 6” should be understood to include reference to all forms and components of Hev b 6 or derivatives, mutants, homologues, analogues, equivalents or mimetics thereof. For example, it includes reference to Hev b 6.02 (also known as “Hevein”). Hevein is a 4.7 kDa hydrophilic protein which forms the N-terminus of pro-hevein and has been found, on skin testing, to react with 81% of patients with latex allergy (Chen Z et al., 1997; J. Allergy Clin Immunol., 99(3):402-9). Reference to Hev b 6 should also be understood to include reference to pro-hevein (also known as Hev b 6.01) which is a 20 kDa protein and to Hev b 6.03 which is a 14 kDa protein. Hev b 6 has been shown to share homology with Class 1 chitinases, which occur widely in plants. Accordingly, Hev b 6 is characterised as a “pan allergen”. Reference to “Hev b 6” should also be understood to include reference to all protein forms of Hev b 6 or its functional equivalent or derivative including, for example, any isoforms which may arise from alternative splicing of Hev b 6 mRNA. It includes reference to mutants, polymorphic variants or homologues of Hev b 6, such as the homologous Class 1 chitinase proteins detailed above. It also includes reference to analogues or equivalents of Hev b 6 such as may occur where a product which naturally comprises Hev b 6 is synthetically generated for the purpose of generating a product such as gloves. The present invention thereby provides epitopes and methods for their use in the diagnosis and treatment of any condition characterised by hypersensitivity to a Hev b 6 or Hev b 6-like molecule such as latex allergy or a plant allergy. Preferably, said Hev b 6 comprises the sequence set forth in SEQ ID NO:1 or is a derivative, homologue, analogue, chemical equivalent, mutant or mimetic of said sequence.

The present invention therefore more particularly provides an isolated peptide of the formula:
X₁X₂X₃
wherein:
X₁ and X₃ may be the same or different and each is an amino acid sequence comprising from 0 to 40 naturally or non-naturally occurring amino acid residues;
X₂ is an amino acid sequence of from 5 to 100 residues derived from, homologous to or contiguous with amino acids 1-187 inclusive or derivatives thereof of Hev b 6;
   and wherein said peptide molecule is capable of interacting with T cells and modifying T cell function when incubated with cells from subjects having a condition characterised by an aberrant, unwanted or otherwise inappropriate immune response to Hev b 6 or a derivative, homologue, mutant, chemical equivalent or mimetic of said peptide.

Still more particularly the present invention provides an isolated peptide of the formula:
X₁X₂X₃
wherein
X₁ and X₃ may be the same or different and each is an amino acid sequence comprising from 0 to 40 naturally or non-naturally occurring amino acid residues;
X₂ is an amino acid sequence of from 5 to 100 residues derived from, homologous to or contiguous with amino acids 1-47, 55-83, 81-101 and/or 136-155 inclusive or derivatives thereof of Hev b 6;
   and wherein said peptide molecule is capable of interacting with T cells and modifying T cell function when incubated with cells from subjects having a condition characterised by an aberrant, unwanted or otherwise inappropriate immune response to Hev b 6 or a derivative, homologue, analogue, mutant, chemical equivalent or mimetic of said peptide.

Still more particularly, X₂ is any amino acid sequence of from 5 to 100 residues derived from, homologous to or contiguous with amino acids 1-20, 10-29, 19-38, 28-47, 55-74, 64-83 and/or 136-155 inclusive or derivatives thereof of Hev b 6.

Yet more particularly, X₂ is any amino acid sequence of from 5 to 100 residues derived from homologous to or contiguous with amino acids 1-20, 10-29, 19-38 and/or 2847 inclusive or derivatives thereof of Hev b 6.

Most particularly, X₂ is any amino acid sequence of from 5 to 100 residues derived from, homologous to or contiguous with amino acids 10-29 and/or 19-38 inclusive or derivatives thereof of Hev b 6.

Reference to “T cells” should be understood as a reference to any cell comprising a T cell receptor. In this regard, the T cell receptor may comprise any one or more of the α, β, γ or δ chains. The present invention is not intended to be limited to any particular functional sub-class of T cells although in a preferred embodiment the subject T cell is a T helper cell and still more preferably a Th2-type cell, predominantly. In this regard, reference to “modifying T cell function” should be understood as a reference to modifying any one or more functions which a T cell is capable of performing. For example, the subject function may be proliferation, differentiation or other form of cellular functional activity such as the production of cytokines. Preferably, the subject functional activity is proliferation.

In terms of modifying the function of T cells from subjects having a condition characterised by an aberrant, unwanted or inappropriate immune response to Hev b 6, it should be understood that this is not necessarily a reference to modifying the function of all the T cells in a given sample but is likely, in fact, to reflect the modification or functioning of only some of the T cells in the sample. For example, only a portion of the T helper cells in a given T cell sample may functionally respond to contact with the subject peptide. Such a partial response should be understood to fall within the scope of the present invention. It should also be understood that the T cells which are derived from the subject may be freshly harvested T cells or they may have undergone some form of in vitro or in vivo manipulation prior to testing. For example, T cell lines may have been generated from the cell sample and it is these T cell lines which then form the subject derived T cell population which is tested in accordance with the present invention. To the extent that the subject functional activity is T cell proliferation, the T cell proliferation assay is preferably performed as disclosed herein. Still more preferably, the subject modification of T cell function is the induction of a proliferation index of ≧2.5.

Reference to an “aberrant, unwanted or otherwise inappropriate” immune response should be understood as a reference to any form of physiological activity which involves the activation and/or functioning of one or more immune cells where that activity is inappropriate in that it is of an inappropriate type or proceeds to an inappropriate degree. It may be aberrant in that according to known immunological principals it either should not occur when it does so or else should occur when it does not do so. In another example, the immune response may be inappropriate in that it is a physiologically normal response but which is unnecessary and/or unwanted, such as occurs with respect to type-I hypersensitivity responses to innocuous allergens. Preferably said immune response is latex hypersensitivity.

By “latex hypersensitivity” it should be understood to mean the exhibition of clinical symptoms of IgE mediated latex hypersensitivity. However, it should be understood that although clinical symptoms may be evident, not all such individuals would necessarily exhibit detectable levels of latex specific serum IgE which is measured using the Kallestad Allercoat EAST System (Sanofi-Pasteur Diagnostics, USA). In accordance with this test, the latex EAST score in non-allergic individuals is 0/4. Any subject exhibiting a latex EAST score greater than 0/4 should be understood as a subject exhibiting latex hypersensitivity within the context of the present invention although there will also be individuals with latex hypersensitivity who do not exhibit such a score but nevertheless fall within the scope of the invention. Alternatively, testing may proceed utilising either the Pharmacia or the UniCap systems.

In a preferred embodiment, X₂ comprises not less than about 5 and not greater than about 50 amino acid residues, more preferably not less than about 5 and not greater than about 30 amino acid residues and even more preferably not less than about 5 and not greater than about 20.

In a particularly preferred embodiment, X₂ comprises a sequence of at least 5 amino acids derived from one or more of the following amino acid sequences:

| | |
|---|---|
| EQCGRQAGGKLCPNNLCCSQ | (SEQ ID NO:2) |
| KLCPNNLCCSQWGWCGSTDE | (SEQ ID NO:3) |
| SQWGWCGSTDEYCSPDHNCQ | (SEQ ID NO:4) |
| DEYCSPDHNCQSNCKDSGEG | (SEQ ID NO:5) |

More preferably, X₂ comprises a sequence of at least 5 amino acids derived from one or more of SEQ ID NO:3 or SEQ ID NO:4.

Reference to a “peptide” includes reference to a peptide, polypeptide or protein or parts thereof. The peptide may be glycosylated or unglycosylated and/or may contain a range of other molecules fused, linked, bound or otherwise associated to the protein such as amino acids, lipids, carbohydrates or other peptides, polypeptides or proteins. Reference hereinafter to a “peptide” includes a peptide comprising a sequence of amino acids as well as a peptide associated with other molecules such as amino acids, lipids, carbohydrates or other peptides, polypeptides or proteins.

“Derivatives” include fragments, parts, portions and variants from natural, synthetic or recombinant sources including fusion proteins. Parts or fragments include, for example, active regions of the subject peptide. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence.

Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins.

Chemical and functional equivalents of the subject peptide should be understood as molecules exhibiting any one or more of the functional activities of these molecules and may be derived from any source such as being chemically synthesized or identified via screening processes such as natural product screening.

Homologues include peptides derived from species other than Hevea brasiliensis, such as peptides derived from kiwi fruit.

The derivatives include fragments having particular epitopes or parts of the entire protein fused to peptides, polypeptides or other proteinaceous or non-proteinaceous molecules.

Analogues contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues. Mutants include molecules which exhibit modified functional activity (for example, Hev b 6 peptides which express one or more T cell epitopes but lack B cell reactivity).

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 3.

TABLE 3

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

It is possible to modify the structure of a peptide according to the invention for various purposes such as for increasing solubility, enhancing therapeutic or preventative efficacy, enhancing stability or increasing resistance to proteolytic degradation. A modified peptide may be produced in which the amino acid sequence has been altered, such as by amino acid substitution, deletion or addition, to modify immunogenicity and/or reduce allergenicity. Similarly components may be added to peptides of the invention to produce the same result.

For example, a peptide can be modified so that it exhibits the ability to induce T cell anergy. In this instance, critical binding residues for the T cell receptor can be determined using known techniques (for example substitution of each residue and determination of the presence or absence of T cell reactivity). In one example, those residues shown to be essential to interact with the T cell receptor can be modified by replacing the essential amino acid with another, preferably similar amino acid residue (a conservative substitution) whose presence is shown to alter T cell reactivity or T cell functioning. In addition, those amino acid residues which are not essential for T cell receptor interaction can be modified by being replaced by another amino acid whose incorporation may then alter T cell reactivity or T cell functioning but does not, for example, eliminate binding to relevant MHC proteins. In yet another example, mutant peptides may be created which exhibit normal T cell binding but abrogated IgE binding.

Such modifications will result in the production of molecules falling within the scope of “mutants” of the subject peptide as herein defined. “Mutants” should be understood as a reference to peptides which exhibit one or more structural features or functional activities which are distinct from those exhibited by the non-mutated peptide counterpart.

Peptides of the invention may also be modified to incorporate one or more polymorphisms resulting from natural allelic variation and D-amino acids, non-natural amino acids or amino acid analogues may be substituted into the peptides to produce modified peptides which fall within the scope of the invention. Peptides may also be modified by conjugation with polyethylene glycol (PEG) by known techniques. Reporter groups may also be added to facilitate purification and potentially increase solubility of the peptides according to the invention. Other well known types of modification including insertion of specific endoprotease cleavage sites, addition of functional groups or replacement of hydrophobic residues with less hydrophobic residues as well as site-directed mutagenesis of DNA encoding the peptides of the invention may also be used to introduce modifications which could be useful for a wide range of purposes. The various modifications to peptides according to the invention which have been mentioned above are mentioned by way of example only and are merely intended to be indicative of the broad range of modifications which can be effected.

In related aspects, the method of the present invention provides a mutant form of the peptides hereinbefore defined wherein said peptide molecule retains all or some of its capacity to interact with T cells but exhibits partially or completely inhibited, abrogated or otherwise down-regulated antibody reactivity. Effecting the down-regulation of antibody reactivity can be achieved by any suitable method, which methods would be well known to those skilled in the art. For example, to the extent that a B cell epitope is defined by its linear amino acid sequence, one may add, delete or substitute one or more amino acid residues in order to render the mutated linear sequence distinct from the naturally occurring sequence. To the extent that the epitope is additionally, or alternatively, defined by a conformation epitope, one may seek to disrupt that conformation by disrupting the 2° or, to the extent that homodimers or heterodimers exist, the 3° structure of the peptide. This may be achieved, for example, by disrupting the formation of bonds, such as disulphide bonds, which stabilise the 2° and/or 3° structure.

In the context of the present invention, and in a particularly preferred embodiment, to the extent that the subject peptide comprises a contiguous sequence of amino acids derived from residues 1-43 of SEQ ID NO: 1, the formation of any one or more disulphide bonds is disrupted. Without limiting the present invention to any one theory or mode of action, amino acid residues 1-43 of SEQ ID NO: 1 correspond to the Hevein region of Hev b 6 and, in is naturally occurring 2° structure, comprises 4 disulphide bonds.

Accordingly, in a related aspect the present invention provides a peptide, as hereinbefore defined, wherein the antibody reactivity of said peptide is inhibited, abrogated or otherwise down-regulated.

More preferably, said antibody is IgE. Even more preferably, said down-regulation is achieved by disrupting the formation of any one or more disulphide bonds. Most preferably, said disulphide bond is the bond associated with the cysteine residue at position 3 of SEQ ID NO: 1. In a particularly preferred embodiment, the disulphide bond which forms with the cysteine amino acid at position 3 of SEQ ID NO: 1 is disrupted.

Another aspect of the present invention provides an isolated peptide comprising any amino acid sequence derived from or homologues to Hev b 6 wherein said peptide molecule is capable of interacting with T cells and modifying T cell function when incubated with cells from subjects having a condition characterised by an aberrant, unwanted or otherwise inappropriate immune response to Hev b 6 or a derivative, homologue, analogue, mutant, chemical equivalent or mimetic of said peptide.

More particularly, the present invention provides an isolated peptide comprising an amino acid sequence of from 5-100 residues derived from, homologues to or contiguous with amino acids 1-187 inclusive or derivatives thereof of Hev b 6 wherein said peptide molecule is capable of interacting with T cells and modifying T cell function when incubated with cells from subjects having a condition characterised by an aberrant, unwanted or otherwise inappropriate immune response to Hev b 6 or a derivative, homologue, analogue, mutant, chemical equivalent or mimetic of said peptide.

In one preferred embodiment said amino acid sequence is derived from, homologous to or contiguous with amino acids 1-47, 55-83 and/or 136-155 inclusive or derivatives thereof of Hev b 6.

In another preferred embodiment said amino acid sequence is derived from, homologous to or contiguous with amino acids 1-20, 10-29, 19-38, 28-47, 55-74, 64-83 and/or 136-155 inclusive or derivatives thereof of Hev b 6.

In yet another preferred embodiment said amino acid sequence is derived from, homologous to or contiguous with amino acids 1-20, 10-29, 19-38 and/or 28-47 inclusive or derivatives thereof of Hev b 6.

More preferably, said amino acid sequence is derived from, homologous to or contiguous with amino acids 10-29 and/or 19-38 inclusive or derivatives thereof of Hev b 6.

In another aspect said amino acid sequence comprises a sequence of at least 5 amino acids derived from one or more of the following amino acid sequences:

| | |
|---|---|
| EQCGRQAGGKLCPNNLCCSQ | (SEQ ID NO:2) |
| KLCPNNLCCSQWGWCGSTDE | (SEQ ID NO:3) |
| SQWGWCGSTDEYCSPDHNCQ | (SEQ ID NO:4) |
| DEYCSPDHNCQSNCKDSGEG | (SEQ ID NO:5) |

According to this aspect, said amino acid sequence preferably comprises a sequence of at least 5 amino acids derived from one or more of SEQ ID NO:3 or SEQ ID NO:4.

The peptides of the present invention may be prepared by recombinant or chemical synthetic means. According to a preferred aspect of the present invention, there is provided a recombinant peptide which is preferentially immunologically reactive with T cells from individuals with latex hypersensitivity, which is expressed by the expression of a host cell transformed with a vector coding for the peptide sequence of the present invention. The peptide may be fused to another peptide, polypeptide or protein. Alternatively, the peptide may be prepared by chemical synthetic techniques, such as by the Merrifield solid phase synthesis procedure. Furthermore, although synthetic peptides of the formula given above represent a preferred embodiment, the present invention also extends to biologically pure preparations of the naturally occurring peptides or fragments thereof. By “biologically pure” is meant a preparation comprising at least about 60%, preferably at least about 70%, or preferably at least about 80% and still more preferably at least about 90% or greater as determined by weight, activity or other suitable means.

Still another aspect of the present invention is directed to antibodies to Hev b 6 including catalytic antibodies or derivatives, homologues, analogues, mutants, chemical equivalents or mimetics of said antibodies. Such antibodies may be monoclonal or polyclonal and may be selected from naturally occurring antibodies to Hev b 6 or may be specifically raised to Hev b 6. In the case of the latter, Hev b 6 may first need to be associated with a carrier molecule. The antibodies and/or recombinant Hev b 6 of the present invention are particularly useful as therapeutic or diagnostic agents. Alternatively, fragments of antibodies may be used such as Fab fragments or Fab′₂ fragments. Furthermore, the present invention extends to recombinant and synthetic antibodies and to antibody hybrids. A “synthetic antibody” is considered herein to include fragments and hybrids of antibodies. Hev b 6 can also be used to screen for naturally occurring antibodies to Hev b 6.

Both polyclonal and monoclonal antibodies are obtainable by immunization with Hev b 6 or derivative, homologue, analogue, mutant, chemical equivalent or mimetic thereof and either type is utilizable for immunoassays. The methods of obtaining both types of sera are well known in the art. Polyclonal sera are less preferred but are relatively easily prepared by injection of a suitable laboratory animal with an effective amount of Hev b 6, or antigenic parts thereof, collecting serum from the animal, and isolating specific sera by any of the known immunoadsorbent techniques. Although antibodies produced by this method are utilizable in virtually any type of immunoassay, they are generally less favoured because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See, for example Douillard and Hoffman, Basic Facts about Hybridomas, in Compendium of Immunology Vol II, ed. by Schwartz, 1981; Kohler and Milstein, Nature 256: 495-499, 1975; European Journal of Immunology 6: 511-519, 1976).

Preferably, the antibody of the present invention specifically binds Hev b 6 or derivative, homologue, analogue, mutant, chemical equivalent or mimetic thereof. By “specifically binds” is meant high avidity and/or high affinity binding of an antibody to a specific antigen. Antibody binding to its epitope on this specific antigen is stronger than binding of the same antibody to any other epitope, particularly those that may be present in molecules in association with, or in the same sample, as the specific antigen of interest. Antibodies that bind specifically to a polypeptide of interest may be capable of binding other polypeptides at a weak, yet detectable, level (e.g., 10% or less of the binding shown to the polypeptide of interest). Such weak binding, or background binding, is readily discernible from the specific antibody binding to the polypeptide of interest, e.g. by use of appropriate controls.

In a preferred embodiment, the subject antibody is expressed by hybridoma 1A5.4 or 6E5.3 or derivative, homologue, analogue, chemical equivalent, mutant or mimetic thereof.

The hybridoma cell line secreting 1A5.4.3 was deposited with ECACC on 21 Dec. 2001 under Accession Number 01122118.

The development of monoclonal antibodies to Hev b 6 now permits the identification, isolation and synthesis of Hev b 6 B cell epitopes and the present invention should accordingly be understood to extend to Hev b 6 B cell epitopes and to derivatives, homologues, analogues, mutants, chemical equivalents or mimetics thereof.

In one preferred embodiment there is provided an isolated peptide of the formula:
X₁X₂X₃
wherein
X₁ and X₃ may be the same or different and each is an amino acid sequence comprising from 0 to 40 naturally or non-naturally occurring amino acid residues;
X₂ is an amino acid sequence derived from, or homologous to Hev b 6
   and wherein said peptide molecule is capable of interacting with antibody from subjects having a condition characterised by an aberrant, unwanted or otherwise inappropriate immune response to Hev b 6 or a derivative, homologue, analogue, mutant, chemical equivalent or mimetic of said peptide.

Preferably, said condition is latex hypersensitivity and said antibodies are of the IgE isotype.

More particularly the present invention provides an isolated peptide of the formula:
X₁X₂X₃
wherein
X₁ and X₃ may be the same or different and each is an amino acid sequence comprising from 0 to 40 naturally or non-naturally occurring amino acid residues;
X₂ is any amino acid sequence derived from, or homologous to Hev b 6
   and wherein said peptide molecule is capable of interacting with antibodies expressed by hybridoma 1A5.4 or 6E5.3 or a derivative, homologue, analogue, mutant, chemical equivalent or mimetic of said peptide.

The present invention also extends to hybridomas or variants or mutants thereof which express the antibodies hereinbefore defined.

Reference to “Hev b 6”, and “peptide” should be understood to have the same meaning as hereinbefore defined. Similarly, reference to a condition involving an “aberrant, unwanted or otherwise inappropriate” immune response and “latex hypersensitivity” should also be understood to have the same meaning as hereinbefore provided. Further, reference to “derivatives, homologues, analogues, chemical equivalents and mimetics of the subject peptide or antibody also has the same meaning as provided earlier. The peptides encompassed by this aspect of the present invention may also undergo modification as hereinbefore detailed. Such modification is particularly useful for generating mutant peptides which are useful for a prophylactic and/or therapeutic treatment of individuals suffering from or predisposed to a condition characterised by an aberrant, unwanted or otherwise inappropriate immune response to Hev b 6. For example, it may be particularly useful to generate a mutant peptide comprising T cell epitopic regions but which peptides lack B cell epitopes capable of interacting with IgE. Such peptides may be generated by synthesising peptides comprising only T cell epitopes or by mutating naturally occurring molecules such that the T cell epitopes remain functional while the B cell epitopes are altered to prevent antibody binding. It should also be understood that the subject “amino acid sequence” may correspond to the sequence of either a continuous or discontinuous epitope of Hev b 6.

The present invention should therefore be understood to encompass peptides that comprise at least one B or T cell epitope of Hev b 6 in conjunction with other amino acids (which may or may not be naturally occurring as amino acid analogues) or other chemical species. In a preferred aspect of the invention such peptides may comprise one or more epitopes of Hev b 6, which epitopes may be T or B cell epitopes. Peptides with one or more epitopes of Hev b 6 are desirable for increased therapeutic effectiveness.

In another aspect, the present invention provides a nucleic acid molecule comprising a sequence of nucleotides encoding or complementary to a sequence encoding the peptides as hereinbefore defined or a derivative, homologue or analogue thereof. It should be understood that reference to “peptides” includes reference to peptides comprising one or more T cell epitopes, one or more B cell epitopes or a combination of B and T cell epitopes. A nucleic acid molecule encoding the subject peptide is preferably a sequence of deoxyribonucleic acids such as cDNA or a genomic sequence. A genomic sequence may comprise exons and introns. A genomic sequence may also include a promoter region or other regulatory regions.

The nucleic acid molecule may be ligated to an expression vector capable of expression in a prokaryotic cell (eg. E. coli) or a eukaryotic cell (eg. yeast cells, fungal cells, insect cells, mammalian cells or plant cells). The nucleic acid molecule may be ligated or fused or otherwise associated with a nucleic acid molecule encoding another entity such as, for example, a signal peptide. It may also comprise additional nucleotide sequence information fused, linked or otherwise associated with it either at the 3′ or 5′ terminal portions or at both the 3′ and 5′ terminal portions. The nucleic acid molecule may also be part of a vector, such as an expression vector. The latter embodiment facilitates production of recombinant forms of the subject peptide which forms are encompassed by the present invention.

Such nucleic acids may be useful for recombinant production of T cell epitopes of Hev b 6 or proteins comprising them by insertion into an appropriate vector and transfection into a suitable cell line. Such expression vectors and host cell lines also form an aspect of the invention.

In producing peptides by recombinant techniques, host cells transformed with a nucleic acid having a sequence encoding a peptide according to the invention or a functional equivalent of the nucleic acid sequence are cultured in a medium suitable for the particular cells concerned. Peptides can then be purified from cell culture medium, the host cells or both using techniques well known in the art such as ion exchange chromatography, gel filtration chromatography, ultrafiltration, electrophoresis or immunopurification with antibodies specific for the peptide.

Nucleic acids encoding Hev b 6 or peptides comprising T and/or B cell epitopes of Hev b 6 may be expressed in bacterial cells such as E. coli, insect cells, yeast or mammalian cells such as Chinese hamster ovary cells (CHO). Suitable expression vectors, promoters, enhancers and other expression control elements are referred to in Sambruck et al; “Molecular cloning: a laboratory manual, Second Edition”; Cold Spring Harbour Laboratory Press; Cold Spring Harbour, N.Y.; 1989. Other suitable expression vectors, promoters, enhancers and other expression elements are well known to those skilled in the art. Examples of suitable expression vectors in yeast include Yep Sec I (Balderi et al., 1987); pMFa (Kurjan and Herskowitz., 1982; Cell., 30:933-943); JRY88 (Schultz et al., 1987; Gene., 54:113-123) and pYES2 (Invitrogen Corporation, San Diego, Calif.). These vectors are freely available as are baculovirus and mammalian expression systems. For example, a baculovirus system is commercially available (ParMingen, San Diego, Calif.) for expression in insect cells while the pMsg vector is commercially available (Pharmacia, Piscataway, N.J.) for expression in mammalian cells.

For expression in E. coli suitable expression vectors include among others, pTrc (Amann et al, 1988) pGex (Amrad Corporation, Melbourne, Australia); pMal (N.E. Biolabs, Beverley, Mass.); pRit5 (Pharmacia, Piscataway, N.J.); pEt-1 d (Novagen, Maddison, Wis.) (Jameel Jameel et al., 1990; J. Virol., 64:3963-3966) and pSem (Knapp et al., 1990; Bio Techniques., 8:280-281). The use of pTRC, and pEt-11d, for example, will lead to the expression of unfused protein. The use of pMal, pRit5, pSem and pGex will lead to the expression of allergen fused to maltose E binding protein (pMal), protein A (pRit5), truncated -galactosidase (PSEM) or glutathione S-transferase (pGex). When a T cell epitope of Hev b 6 or a peptide comprising it is expressed as a fusion protein, it is particularly advantageous to introduce an enzymatic cleavage site at the fusion junction between the carrier protein and the peptide concerned. The peptide of the invention may then be recovered from the fusion protein through enzymatic cleavage at the enzymatic site and biochemical purification using conventional techniques for purification of proteins and peptides. Examples of enzymatic cleavage sites include those for blood clotting factor Xa or thrombin for which the appropriate enzymes and protocols for cleavage are commercially available. The different vectors also have different promoter regions allowing constitutive or inducible expression or temperature induction. It may additionally be appropriate to express recombinant peptides in different E. coli hosts that have an altered capacity to degrade recombinantly expressed proteins. Alternatively, it may be advantageous to alter the nucleic acid sequence to use codons preferentially utilised by E. coli, where such nucleic acid alteration would not effect the amino acid sequence of the expressed proteins.

Host cells can be transformed to express the nucleic acids of the invention using conventional techniques such as calcium phosphate or calcium chloride co-precipitation, DEAE-dextran-mediated transfection or electroporation. Suitable methods for transforming the host cells may be found in Sambruck et al 1989 (supra), and other laboratory texts. The nucleic acid sequence of the invention may also be chemically synthesised using standard techniques.

In addition to recombinant production of peptides according to the invention, the nucleic acids may be utilised as probes for experimental or purification purposes.

The identification of both B and T cell epitopic regions facilitates the identification and/or rational design of a range of mutant peptide molecules. As detailed hereinbefore, these mutant peptides may comprise one or more mutated B cell epitopes. However, in accordance with the antibody/B cell epitope related aspect of the present invention, there is provided scope for the generation of mutant peptides comprising mutated B cell epitopes or combinations of intact versus mutated B and T cell epitopes. The applications of these molecules are described in more detail below but in a preferred embodiment relate to modulation of the Hev b 6 hypersensitivity immune response in terms of either a prophylactic or therapeutic treatment.

Identification and synthesis of the Hev b 6 T and B cell epitopes and generation of Hev b 6 antibodies as disclosed herein now facilitates the development of a range of diagnostic and prophylactic/therapeutic treatment protocols for use with respect to Hev b 6 related immune conditions. Also facilitated is the development of reagents for use therein.

Accordingly, the present invention should be understood to extend to the use of the peptides and monoclonal antibodies or derivatives, homologues, analogues, mutants, chemical equivalents or mimetics thereof of the present invention in the therapeutic and/or prophylactic treatment of patients. Such methods of treatment include, but are not limited to:
(i) Administration of the subject peptides to a patient as a means of desensitising or inducing immunological tolerance to Hev b 6 or Hev b 6-like molecules. This may be achieved, for example, by inducing Hev b 6 directed Th2 anergy or apoptosis. Such an outcome may be achieved by any one of a number of techniques including the use of peptides which maintain T cell epitope reactivity but which either naturally or as a result of mutation are unable to undergo IgE binding. Alternatively, one may utilise desensitisation/treatment protocols which are based on the administration of specific concentrations of a given peptide in accordance with a specific regime in order to induce tolerance. Such methodology may eliminate Hev b 6 hypersensitivity or it may reduce the severity of Hev b 6 hypersensitivity.
   Preferably such treatment regimes are capable of modifying the T cell response or both the B and T cell response of the individual concerned. As used herein, modification of the allergic response of the individual suffering from Hev b 6 hypersensitivity can be defined as inducing either non-responsiveness or diminution in symptoms to the Hev b 6 molecule as determined by standard clinical procedures (Varney et al., 1990., British Medical Journal., 302:265-269). Diminution in the symptoms includes any reduction in an allergic response in an individual to Hev b 6 after a treatment regime has been completed. This diminution may be subjective or clinically determined, for example by using standard skin tests known in the art.
   Exposure of an individual to the peptides of the present invention, which peptides comprise at least one T cell epitope, may tolerise or anergise appropriate T cell subpopulations such that they become unresponsive to Hev b 6 and do not participate in stimulating an immune response upon such exposure. Preferably the peptides according to the invention will retain immunodominent T cell epitopes but possess abrogated IgE binding.
   Administration of a peptide of the invention may modify the cytokine secretion profile as compared with exposure to naturally occurring Hev b 6 allergen. This exposure may also influence T cell subpopulations which normally participate in the allergic response to migrate away from the site or sites of normal exposure to the allergen and towards the site or sites of therapeutic administration. This redistribution of T cell subpopulations may ameliorate or reduce the ability of an individual's immune system to stimulate the usual immune response at the site of normal exposure to the allergen, resulting in diminution of the allergic symptoms.
   Modification of the B cell response may be achieved, for example, via modulation of the cytokine profile produced by T cells, as detailed above. Specifically, decreasing T cell derived IL-4 and IL-13 production thereby decreasing IgE synthesis.
(ii) The peptides of the present invention may be used in the capacity of an adsorbent to remove Hev b 6 directed antibodies and/or T cells from a biological sample or from a patient.
(iii) The antibodies generated in accordance with the method of the present invention following humanisation or modification to produce Fabs, for example, may be used to abrogate or decrease Hev b 6 hypersensitivity via the administration to an individual of antibodies which bind Hev b 6 thereby competitively inhibiting binding of Hev b 6 to IgE coated mast cells, which latter binding would lead to mast cell degranulation and the onset of hypersensitivity symptoms. Although such a mechanism is unlikely to lead to long term desensitisation, it nevertheless provides a mechanism for preventing or reducing acute hypersensitivity symptom severity. This is likely to be of particular use to individuals who suffer from severe symptoms following certain types of exposure to Hev b 6, such as airway constriction.

Accordingly, in another aspect the present invention provides a method for the treatment and/or prophylaxis of a condition in a subject, which condition is characterised by the aberrant, unwanted or otherwise inappropriate immune response to Hev b 6, said method comprising administering to said subject an effective amount of a peptide and/or antibody as hereinbefore defined for a time and under conditions sufficient to remove or reduce the presence or function in said subject of T cells and/or antibodies directed to said Hev b 6.

Preferably said condition is latex hypersensitivity.

An “effective amount” means an amount necessary at least partly to attain the desired immune response, or to delay the onset or inhibit progression or halt altogether, the onset or progression of a particular condition being treated. The amount varies depending upon the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials.

The subject of the treatment or prophylaxis is generally a mammal such as but not limited to human, primate, livestock animal (e.g. sheep, cow, horse, donkey, pig), companion animal (e.g. dog, cat), laboratory test animal (e.g. mouse, rabbit, rat, guinea pig, hamster), captive wild animal (e.g. fox, deer). Preferably the mammal is a human or primate. Most preferably the mammal is a human. Although the present invention is exemplified using a murine model, this is not intended as a limitation on the application of the present invention to other species, in particular, human.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

Administration of the peptide and/or antibody of the present invention (herein referred to as “agent”) in the form of a pharmaceutical composition, may be performed by any convenient means. The agent of the pharmaceutical composition is contemplated to exhibit therapeutic activity when administered in an amount which depends on the particular case. The variation depends, for example, on the human or animal and the agent chosen. A broad range of doses may be applicable. Considering a patient, for example, from about 0.1 mg to about 1 mg of an agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The agent may be administered in a convenient manner such as by the oral, intravenous (where water soluble), intraperitoneal, intramuscular, subcutaneous, intradermal, intranasal, sublingual or suppository routes or implanting (e.g. using slow release molecules). The agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

Another aspect of the present invention contemplates the use of an agent as hereinbefore defined in the manufacture of a medicament for the treatment of a condition in a mammal, which condition is characterised by an aberrant, unwanted or otherwise inappropriate immune response to Hev b 6.

Preferably said condition is latex hypersensitivity.

In yet another further aspect, the present invention contemplates a pharmaceutical composition comprising an agent as hereinbefore defined and one or more pharmaceutically acceptable carriers and/or diluents. Said agents are referred to as the active ingredients.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding a modulatory agent. The vector may, for example, be a viral vector.

Yet another aspect of the present invention relates to agents, as hereinbefore defined, when used in the method of the present invention.

In yet another aspect, the present invention should be understood to extend to the use of the peptides and/or antibodies of the present invention in diagnostic applications. Said diagnostic applications include, but are not limited to:
(i) To measure the reactivity of a subject's cells to Hev b 6. This is of use, for example, with respect to the diagnosis and/or monitoring of conditions characterised by an aberrant, unwanted or otherwise inappropriate immune response to Hev b 6. The peptides may be added into solution or bound to a solid support together with cells derived from peripheral blood or from tissue biopsies either unfractionated, fractionated or derived as a continuous cell line. Reactivity to the subject peptide may then be measured by standard proliferation assays such as incorporation of H³-thymidine, measurement of expressed or secreted molecules such as surface markers, cytokines or other standard assays of cellular activity which are well known in the art.
(ii) The use of T cell epitope comprising peptides together with a T cell proliferation assay which utilises a T cell sample derived from the subject will facilitate, for example, the identification of a T cell responsive population.
   B cell epitope comprising peptides can be utilised to screen for the presence of antibody at the qualitative and/or quantitative levels.
(iii) The antibodies generated in accordance with the present invention may be utilised as a diagnostic tool for screening for the presence of molecules comprising Hev b 6 cell epitopes in a sample. Said sample may be a biological sample, such as where it is suspected that an individual may have ingested Hev b 6 comprising material. For example, detection of Hev b 6 homologues in patients suffering from suspected fruit hypersensitivity. In another example, it may be desirable to test non-biological samples, such as those suspected of inducing hypersensitivity responses in some individuals who contact these samples, for the presence of Hev b 6. For example, it may be desirable to test rubber gloves in order to assess their qualitative or quantitative Hev b 6 composition.
   The methods which can be utilised to screen for Hev b 6 on the basis of B cell epitope detection using antibody molecules are well known to those skilled in the art and include radio-allergosorbent test (RAST), paper radio immunoabsorbent test (PRIST), enzyme linked immunoabsorbent assay (ELISA), radio-immunoassay (RIA), immunoradiometric assay (IRMA), luminescence immunoassay (LIA), histamine release assays and IgE immunoblots.
   As detailed above, techniques for the assays contemplated herein are known in the art and include, for example, sandwich assays, ELISA and flow cytometry. It is within the scope of this invention to include any second antibodies (monoclonal, polyclonal or fragments of antibodies) directed to the first mentioned antibodies discussed above. Both the first and second antibodies may be used in detection assays or a first antibody may be used with a commercially available anti-immunoglobulin antibody. An antibody as contemplated herein includes any antibody specific to any region of Hev b 6.
   To the extent that antibody based methods of diagnosis are used, the presence of Hev b 6 may be determined in a number of ways such as by Western blotting, ELISA or flow cytometry procedures. These, of course, include both single-site and two-site or “sandwich” assays of the non-competitive types, as well as in the traditional competitive binding assays. These assays also include direct binding of a labelled antibody to a target.
   Sandwich assays are among the most useful and commonly used assays and are favoured for use in the present invention. A number of variations of the sandwich assay technique exist, and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent.
   In the typical forward sandwich assay, a first antibody having specificity for the Hev b 6 or antigenic parts thereof, is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes) and under suitable conditions (e.g. 25° C.) to allow binding of any subunit present in the antibody. Following the incubation period, the antibody subunit solid phase is washed and dried and incubated with a second antibody specific for a portion of the hapten. The second antibody is linked to a reporter molecule which is used to indicate the binding of the second antibody to the hapten. An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.
   By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.
   In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. “Reporter molecule” also extends to use of cell agglutination or inhibition of agglutination such as red blood cells on latex beads, and the like.
   Alternately, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength the fluorescence observed indicates the presence of the hapten of interest. Immunofluorescene and EIA techniques are both very well established in the art and are particularly preferred for the present method. However, other reporter molecules, such as radioisotope, chemiluminescent or bioluminescent molecules, may also be employed.
(iv) Anti-Hev b 6 antibodies may also be utilised as a means of purifying Hev b 6 or the T or B cell epitope comprising peptides thereof which have been made by recombinant means or derived from any other source, such as natural products. Such purification techniques may be applied to isolating Hev b 6 proteins from culture supernatant fluid or from natural products, for example.

Methods of detecting Hev b 6 may be utilised, for example, to qualitatively or quantitatively detect Hev b 6 levels. However, these methods may also be utilised to screen for mutations or polymorphisms in Hev b 6 which mutations may result in, for example, loss of T cell and/or B cell reactivity to Hev b 6. These methods may be utilised for the purpose of screening for peptide molecules suitable for use in therapeutically or prophylactically treating an individual suffering from Hev b 6 related hypersensitivity.

Accordingly, yet another aspect of the present invention is directed to a method of diagnosing or monitoring a condition in a mammal, which condition is characterised by an aberrant, unwanted or inappropriate response to Hev b 6, said method comprising screening for Hev b 6 reactive T cells and/or antibodies utilising the peptides hereinbefore defined.

Preferably said condition is latex hypersensitivity.

Still another aspect of the present invention is directed to a method of qualitatively and/or quantitatively detecting Hev b 6, or peptides thereof, in a sample said method comprising screening for said Hev b 6 or peptides thereof utilising an antibody hereinbefore defined.

In another embodiment the present invention provides diagnostic kits for use in the diagnostic methodology hereinbefore defined.

The present invention will now be further described with reference to the following non-limiting Examples.

### EXAMPLE 1

### The Latex Allergen Hev B 6—Human T Cell Epitope Mapping and Production of a Monoclonal Antibody

### Materials and Methods

Human Subjects

Ten latex-allergic glove users (Table 4) and six non-latex allergic atopic control subjects were recruited from the Alfred Hospital Allergy and Asthma Clinic. The study was approved by the Alfred Hospital Ethics Committee and informed written consent was obtained from all subjects. All latex-allergic subjects had clinical symptoms of IgE-mediated latex hypersensitivity and a grade 34 score of latex-specific serum IgE (Kallestad Allercoat enzyme allergosorbent, EAST, system; Sanofi-Pasteur Diagnostics). Pulversied non-powdered commercial glove extracts are used to manufacture the solid phase latex allergen for this system.

Antigens

Glove Extract: Glove extract was obtained by adding sterile phosphate buffered saline (PBS; Sigma, MO, USA; 1 ml/g glove) to the interior of latex gloves (Uniglove UG2A, made in Malaysia for UNIMEX trade agents P/L, Australia) as described previously (Sutherland M F, Drew A, Rolland J M, Slater J E, Suphioglu C, O'Hehir R E. Specific monoclonal antibodies and human immunoglobulin E show that Hev b 5 is an abundant allergen in high protein powdered latex gloves. Clin Exp Allergy 2002; 32:583-589).

Latex preparations: Low ammoniated latex (LAL) was a gift from Ansell, Melbourne, Australia and non-ammoniated latex (NAL) was obtained from the Rubber Research Institute of Malaysia. Preparations were processed as described previously (Sutherland et al., 2002, supra).

Recombinant Hev b 6 (rHev b 6): The cDNA encoding Hev b 6 was cloned from latex RNA (Beezhold, unpublished). The deduced amino acid sequence of this clone is identical to that of the Hev b 6 clone pDHPROH 6 in Rozynek P et al., 1998, Clinical and Experimental Allergy 28:1418-1426 and differed at amino acid 126 (Thr versus Ala) from the deduced amino acid sequence of the Hev b 6 clone pDHPROH 1-2 (Genbank entry AJ003196.1). It differs at amino acids 109 (Pro versus Ser) and 161 (Leu versus Ile) from the deduced amino acid sequence of the Hev b 6 clone HEV1 (Genbank entry M36986.1), isolated by Broekaert W, Lee H, Kush A, Chua N-H, Raikhel N. Wound-induced accumulation of mRNA containing a hevein sequence in laticifers of rubber tree (Hevea brasiliensis). Proc Natl Acad Sci USA 1990; 87:7633-7637. The Hev b 6 cDNA was subcloned into the pPROEX-Hta vector (Invitrogen, CA, USA) by the polymerase chain reaction (PCR) following our standardised methods (Sutherland et al., 2002, supra). Primers HEVB6F (5′-GCGGAATTCGAGCAATHTGGTCGGCAA-3′) (SEQ ID NO:22) and HEVB6R (5′-GCGGTCGACTTAATTAATTACTGATGATTTCATAACGG-3′) (SEQ ID NO:23) were used for the PCR. The plasmid construct was transformed into E. coli strain BL21-CodonPlus® (DE3)-RIL competent cells (Stratagene, CA, USA) by the heat shock procedure (Sambruk et al., 1989, supra). Recombinant Hev b 6 (rHev b 6) was expressed from the pPROEX-HTa/Hev b 6 construct as previously described (Sutherland et al., 2002, supra) and Ni-NTA affinity purification, under denaturing conditions, was carried out as described by the manufacturer (Qiagen, Victoria, Australia). The rHev b 6 produced under these conditions was insoluble. To improve solubility, purified rHev b 6 was sulphonated by diluting to 1 mg/ml in sulphonation buffer (100 mM Na₂SO₃ 10 mM Na₂O₆S₄.2H₂O, 100 mM Na₂H₂PO₄, 10 mM Tris.Cl, 8 M urea, pH 8.0) and incubating for 2 h at room temperature. Sulphonated rHev b 6 was then dialysed against PBS (pH 7.4) to remove the denaturing and sulphonating reagents.

Hev b 6 peptides: The 43-mer hevein peptide and 20-mer Hev b 6 peptides (overlap of 11 amino acids except for the last two which overlapped by 15; Table 5) were synthesised according to the prohevein amino acid sequence of Rozynek et al. by Mimotopes (Clayton, Victoria, Australia).

Monoclonal Antibody Production

Monoclonal antibody production followed established protocols (Sutherland et al, 2002, supr; Goding, J. Monoclonal Antibodies: principles and practice 3rd ed. London: Academic Press. 492, 1996). Approval was obtained from the Monash University Animal Ethics Committee. Eight BALB/c mice were immunised with 20 μg rHev b 6 in 100 ill of PBS, intraperitoneally, three times at three weekly intervals. The first injection was given with Complete Freund's Adjuvant (100 μl; Sigma) and the two subsequent injections with Incomplete Freund's Adjuvant (100 μl; Sigma). Mice were finally boosted with 20 μg rHev b 6 in 200 μl PBS intraperitoneally 3 days before being sacrificed. Hybridomas were generated by fusing immune mouse spleen cells with murine myeloma cells (line X-63.Ag8.653; 17) and supernatants were screened for antibody to rHev b 6 and glove extract by performing a duplicate ELISA as described below. Positive hybridomas were expanded and cloned by limiting dilution.

ELISA for Hev b 6 Monoclonal Antibody and Latex-Specific Human IgE

Hybridoma supernatants were tested for reactivity to rHev b 6, glove extract and Hev b 6 peptides by ELISA using an established protocol (Sutherland et al., 2002, supra). Patient serum samples were tested for latex-specific IgE using a similar ELISA protocol with rabbit anti-human IgE (1/1000) and swine anti-rabbit immunoglobulins-horseradish peroxidase conjugate (1/1000). Monoclonal antibody isotypes were determined using a mouse immunoglobulin isotyping ELISA kit (BD Biosciences, San Diego, Calif., USA).

Western Blotting for Hev b 6 Monoclonal Antibody and Latex-Specific Human IgE

Hybridoma supernatants and patient sera were tested for reactivity with LAL, NAL, glove extract, rHev b 6 and hevein by 16% SDS-PAGE and immunoblotting as described previously (Sutherland et al., 2002, supra).

Generation of Latex Specific T-Cell Lines

Latex specific T-cell lines were generated using well established methods (De Silva H D, Sutherland M F, Suphioglu C, McLellan S C, Slater J E, Rolland J M, O'Hehir R E. Human T-cell epitopes of the latex allergen Hev b 5 in health care workers. J Allergy Clin Immunol 2000; 105:1017-1024, O'Hehir R E et al., 1987; Immunology., 62:635-640). Briefly peripheral blood mononuclear cells (PBMC) were cultured (2.5×10⁶/well) in 24 well tissue culture plates (Costar, MA, USA) for 7 ays with glove extract at 20 μg/ml, restimulated for another 7 days with glove extract at 20 μg/ml in the presence of an equal number of irradiated (3000 rad) autologous PBMC as antigen presenting cells (APC), and finally for a further 7 days with soluble, sulphonated rHev b 6 at 20 μg/ml with APC. We have previously shown that CD4+ T cells are preferentially expanded in these cultures (O'Hehir et al., 1987, supra) and that T-cell lines generated to the specific allergen from allergic donors have a Th2-type cytokine profile (de Silva et al., 2000, supra).

Oligoclonal T-Cell Proliferation Assays

Oligoclonal T-cell blasts (5×10⁴/well) from the 3-week cultures were incubated in 96-well round bottom plates in triplicate with equal numbers of irradiated autologous PBMC as APC in the presence of glove extract (3, 10, 30 and 100 μg/ml), sulphonated rHev b 6 (3, 10, 30 and 100 μg/ml), or overlapping Hev b 6 peptides (10 and 30 μg/ml). Cultures of T cells and APC in the absence of antigen, in the presence of Lymphocult-T (10 IU/ml; Biotest Folex, Germany), T cells alone, and APC alone were included as controls. After 72 hours, cultures were pulsed with 1 μCi of ³H-thymidine (³HTdR; Amersham, USA) and harvested 12-16 hours later. Proliferation as correlated with ³HTdR incorporation was measured by liquid scintillation spectroscopy. Results are expressed as stimulation indices (cpm of antigen-stimulated T cells divided by cpm of unstimulated T cells) and values≧2.5 were considered positive. Where several concentrations of allergen were tested, values corresponding to the highest proliferative response are shown.

### EXAMPLE 2

### Generation and Characterisation of Hev b 6 Specific Monoclonal Antibodies

All eight mice immunised with rHev b 6 generated a strong polyclonal immune response to rHev b 6 and seven reacted with glove extract by ELISA. Preimmune sera did not react with either rHev b 6 or glove extract. Initial screening of hybridoma supernatants revealed 6 lines that reacted with glove extract. Subsequent expansion and retesting of these hybridomas revealed three lines (1A5, 6E5 and 5C3) which showed strong reactivity with glove extract and rHev b 6. Hybridomas were furthr expanded and subcloned by limiting dilution.

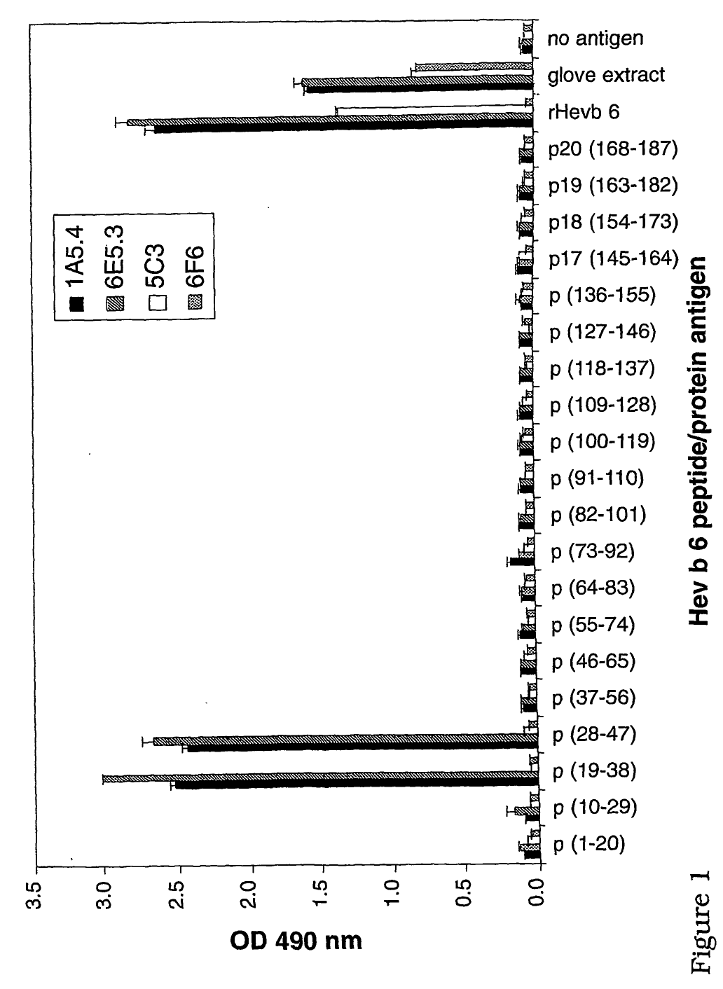

Antibodies produced by all three hybridomas reacted with rHev b 6 and glove extract by ELISA (FIG. 1). 1A5.4 and 6E5.3 also showed strong reactivity to two Hev b 6 peptides, p(19-38) and p(28-47), indicating linear epitopes. A control monoclonal antibody specific for Hev b 5 (6F6; IgG₁/kappa isotype (Sutherland et al., 2002, supra) reacted with the glove extract as expected, but failed to bind rHev b 6 or any of the Hev b 6 peptides. Hydridoma 1A5.4 and 6E5.3 antibody isotypes were IgG₁/kappa, while 5C3 was IgM/kappa.

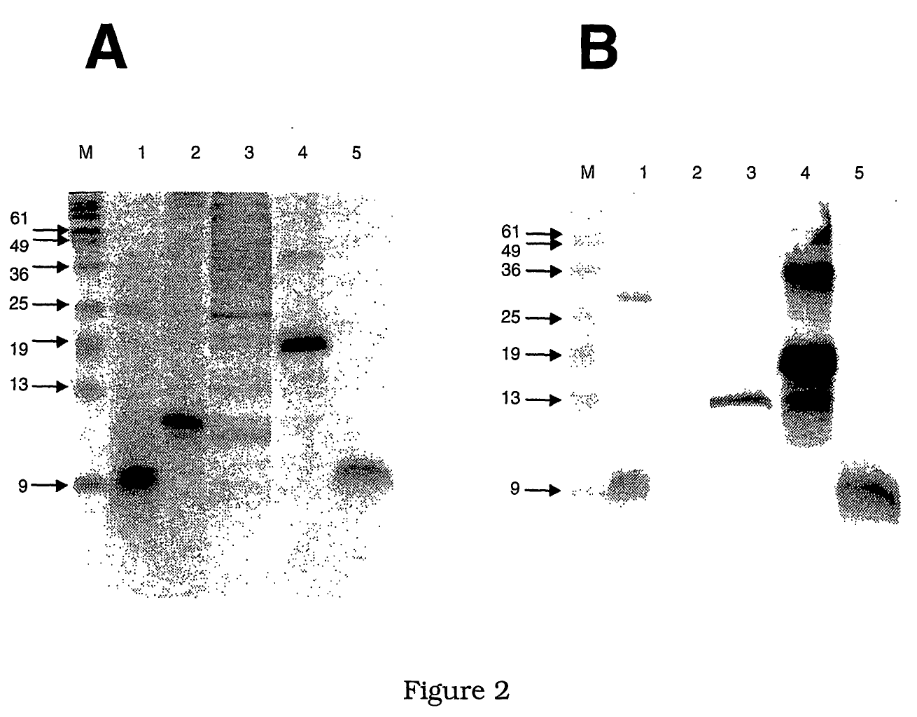

Hybridoma 1A5.4 was further subcloned and expanded and used to characterise the latex extracts. Western blotting revealed strong reactivity of monoclonal antibody (1A5.4.3 to rHev b 6 and hevein (FIG. 2B, lanes 4 and 5). Several bands were detected in the rHev b 6 immunoblot (lane 4), with a strong band at 20 kDa corresponding to that expected for full length prohevein. Other bands were most likely de to reactivity with aggregates and breakdown products of rHev b 6. The monoclonal antibody also reacted strongly with the hevein peptide which migrated at around 10 kDa (lane 5), and predominant reactivity with a band in a similar position was seen for glove extract (lane 1). This band was in fact the major component of the glove extract on the Coomassie-stained gel (FIG. 2A, lane 1). No reactivity with LAL was seen, despite bands being obvious in the Coomassie-stained gel (FIG. 2A, lane 2), but a sharp band at 14 kDa was seen in the NAL immunoblot (lane 3) as seen for rHev b 6 which was most likely an aggregate or fragment containing hevein (lane 4). An isotype control monoclonal antibody (A1, Lo1 p1-specific) showed no reactivity.

### EXAMPLE 3

### Serum IgE Responses to rHev b 6

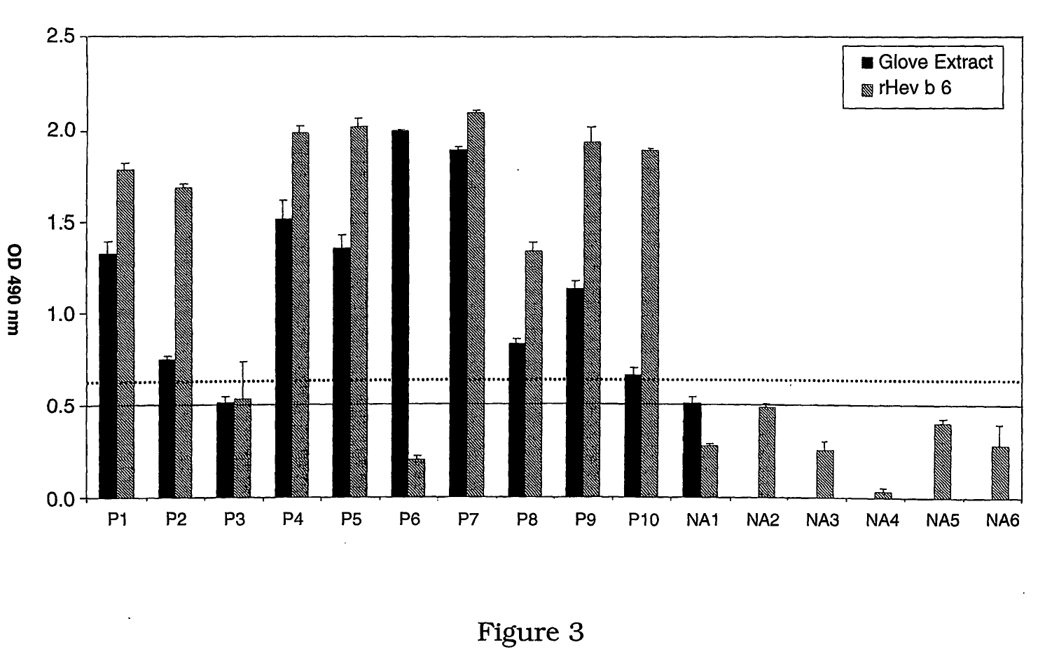

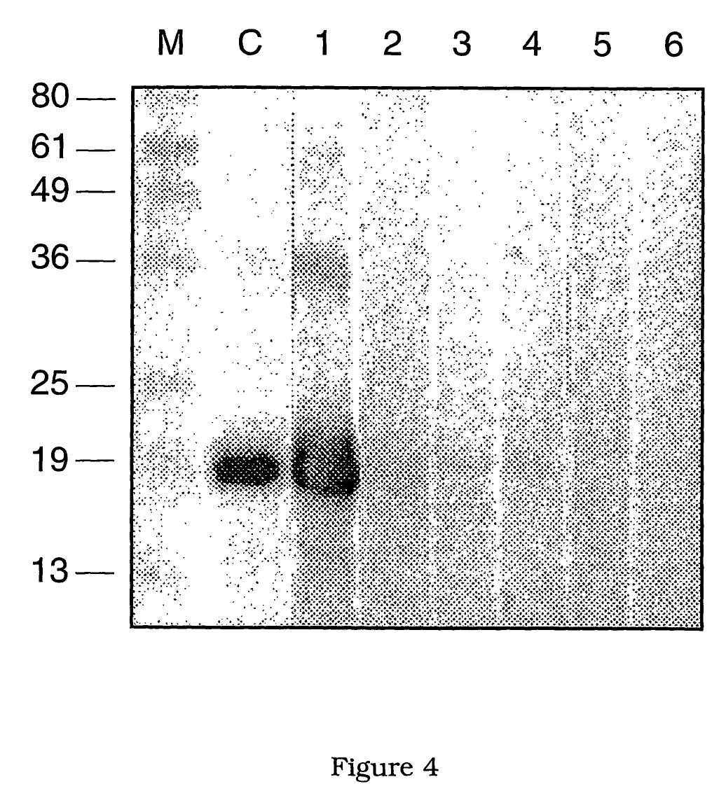

All ten of the latex-allergic patients demonstrated serum IgE binding to glove extract and eight also reacted to rHev b 6 by ELISA (FIG. 3). Sera from patients 1, 4 and 5 were also tested for IgE reactivity with rHev b 6 by Western blotting and all showed reactivity with the prohevein molecule at around 20 kDa (FIG. 4). None of the latex-allergic patients showed serum IgE reactivity to any of the Hev b 6 overlapping peptides by ELISA (data not shown).

### EXAMPLE 4

### T-Cell Responses to Latex Allergens

Since sensitisation to latex in the clinical environment is largely due to latex glove exposure; glove extract was chosen to generate latex-specific T-cell lines in this study. In preliminary primary PBMC cultures, the optimal response to glove extract was observed at 10-30 μg/ml in all cases (data not shown). Therefore glove extract was used at 20 μg/ml to drive the T-cell lines for the first two weeks. In the third week, the latex-specific T-cell lines were stimulated with sulphonated rHev b 6 at 20 μg/ml to enrich for Hev b 6 reactivity. All of the short-term latex-specific T-cell lines generated proliferated in response to rHev b 6 as the whole molecule and/or as one or more peptides (Table 6); eight lines also reacted to glove extract. Mitogenicity and toxicity of all latex allergens and Hev b 6 peptides were excluded.

### EXAMPLE 5

### Hev b 6 T-Cell Epitope Mapping

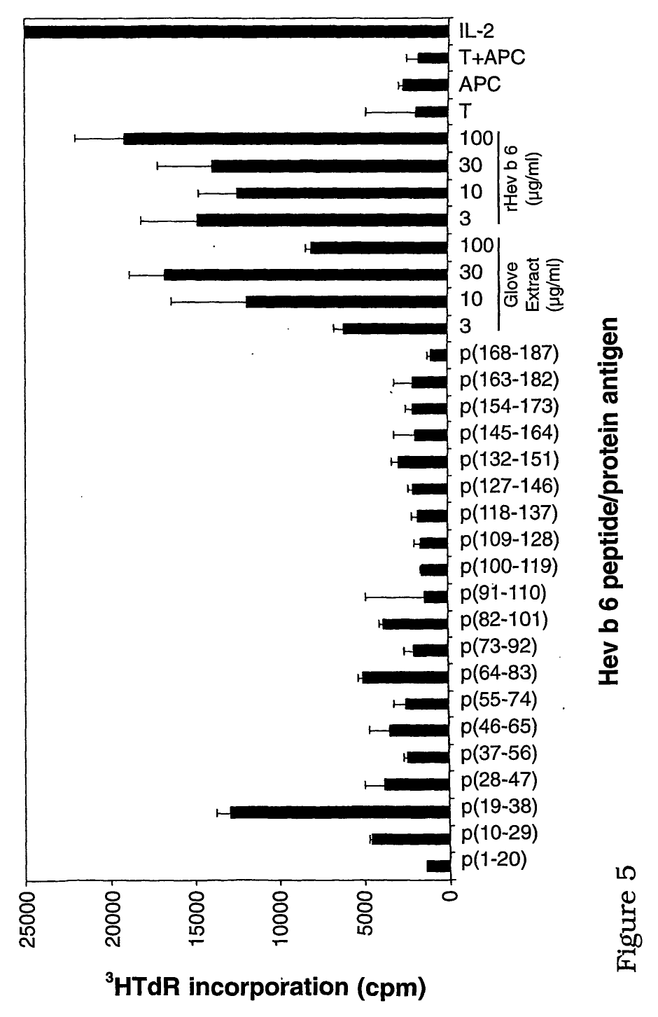

Reactivity to one or more Hev b 6 peptides was identified in the oligoclonal latex-specific T-cell cultures from all of the ten donors. A typical T-cell proliferation assay to the Hev b 6 peptide panel, glove extract and rHev b 6 is shown (FIG. 5). Dominant T-cell responses were identified in the hevein region of the molecule; six patients recognised peptide p(10-29) and seven recognised p(19-38) (Table 6). Four patients showed additional recognition of four peptides in the C-terminal fragment region.

### EXAMPLE 6

### B Cell Epitope Mapping of Hev b 6

Synthetic overlapping peptides based on the deduced amino acid sequence of the mature Hev b 6 protein (twenty 20-mers with 11 amino acid overlap) are be used to identify linear B cell epitopes following established protocols (Suphioglu C et al., 1993; Allergy., 48:273-281). Briefly, synthetic peptides are immobilised either on nitrocellulose membranes or microtitre plates, non-binding sites blocked with 3% BSA/PBS and IgE-binding of patients' sera detected with mouse anti-human IgE followed by horse radish peroxidase conjugated anti-mouse antibodies. Total non-ammoniated latex (NAL) and glove extract (GE) proteins and recombinant Hev b 6 (recHev b 6) are immobilised also, as positive controls. IgE reactive Hev b 6 peptides, along with NAL, GE proteins and recHev b 6, as positive controls, are evaluated for their capacity to inhibit IgE binding to the natural and recHev b 6 allergens. This indicates whether or not the identified IgE-reactive peptides correspond to IgE-reactive epitopes revealed within the context of the natural and recHev b 6 allergens.

Conformational B cell epitopes of Hev b 6 (if any) are identified with random mutagenesis. In this strategy, the plasmid vector containing the cDNA encoding IgE-reactive recombinant Hev b 6 is transformed into XL1-Red host cells (Epicurian Coli, Stratagene, CA, USA) to generate random mutations within the cDNA encoding Hev b 6. The XL1-Red strain is deficient in three of the primary DNA repair pathways in E. coli (ie. mutS, mutD and mutT) making its mutation rate approximately 5,000-fold higher than that of its wild-type parent. After transformation and propagation in XL1-Red strain, plasmid DNA is retrieved and transformed into XL1-Blue strain for analysis. This is necessary as every time the plasmid replicates in the XL1-Red strain, further mutations occur. Once transformed into the XL1-Blue, different dilutions of the transformants are plated to obtain single colonies for immunoscreening with IgE sera (which has been preabsorbed with E. coli extract) known to possess recHev b 6-specific IgE. Immunoscreening of bacterial colonies identifies mutants defective in IgE-binding, as compared to non-mutated recHev b 6 control, and responsible mutations will be resolved by DNA sequencing. Such B cell epitope sequences are compared with the linear B cell epitopes identified above to determine if conformational epitopes have been resolved.

### EXAMPLE 7

### Hypoallergenic Mutant of the Major Latex Glove Allergen Hev b 6, with Retained Human T Lymphocyte Stimulatory Activity

### Material and Methods

Patients

Twelve latex-allergic subjects (A1-A11) were recruited from the Alfred Hospital Allergy and Asthma Clinic (Table 7). The study was approved by the Alfred Hospital Ethics Committee and written informed consent was obtained from each subject. The 10 latex-allergic subjects had clinical symptoms of IgE mediated latex hypersensitivity, and a latex-specific IgE class 2 to 4 (Kallestad Allercoat enzyme allergosorbent, EAST, system; Sanofi-Pasteur Diagnostics). The solid phase latex allergen used in this assay is derived from ground, non-powdered commercial latex gloves. Two atopic patients without latex allergy (N1 and N2) were used as controls (Table 1).

Antigens

Glove extract: Glove extract (GE) was prepared by irrigating latex gloves (Uniglove UG2A, made in Malaysia for UNIMEX trade agents P/L, Australia) with PBS (Sigma, MO, USA; 1 ml/g glove) as described previously (Sutherland et al., 2002, supra).

Recombinant Hev b 6 (rHev b 6): The cDNA encoding Hev b 6 was cloned from latex RNA (Beezhold, unpublished). The deduced amino acid sequence of this clone is identical to that of the Hev b 6 clone pDHPROH 6 in Rozynek et al. 1998, (supra). The Hev b 6 cDNA was subcloned into the pPRoEx-HTa vector (Invitrogen, CA, USA) by the polymerase chain reaction (PCR) following our standardised methods (Sutherland et al., 2002, supra). Primers HEVB6F (5.-GCGGAATTCGAGCAATGTGGTCGGCAAG-3.) (SEQ ID NO:22) and HEVB6R (5.-GCGGTCGACTTAATTAATTACTGATGATTTCATAACGG-3.) (SEQ ID NO:23) were used for the PCR. The plasmid construct was transformed into E. coli strain BL21-CodonPlus® (DE3)-RIL competent cells (Stratagene, CA, USA) by the heat shock procedure (Sambruk et al., 1989, supra). rHev b 6 was expressed from the pPROEX-HTa/Hev b 6 construct as described previously (Sutherland et al., 2002, supra) and Ni-NTA affinity purification, under denaturing conditions, was carried out as described by the manufacturer (QIAGEN, Victoria, Australia). The rHev b 6 produced under these conditions was insoluble.

To improve solubility, purified rHev b 6 was sulphonated by diluting to 1 mg/ml in sulphonation buffer (100 mM Na₂SO₃, 10 mM Na₂O₆S₄.2H₂O, 100 mM Na₂H₂PO₄, 10 mM Tris.Cl, 8 M urea, pH 8.0) and incubating for 2 h at room temperature. Sulphonated rHev b 6 was then dialysed against PBS (pH 7.4) to remove the denaturing and sulphonating reagents.

Refolding was undertaken to produce non-sulphonated soluble rHev b 6. rHev b 6 was diluted to 250 μg/ml in sulphonation buffer and incubated with shaking for 2 h. The sulphonated protein was dialysed against refolding buffer (100 mM Na₂H₂PO₄, 10 mM Tris.Cl, 3 M urea, pH 8.0) to remove the sulphonating reagents. Twelve ml sulphonated rHev b 6 in refolding buffer, was then dialysed O/N at 4° C. against 2.5 L refolding buffer containing 0.2 mM oxidised glutathione (Calbiochem) and 1 mM reduced glutathione (Calbiochem) (De Bernardez Clark E R. Refolding of recombinant proteins. Curr Opin Biotechnol 1997; 9:157-163). Subsequently the solution was dialysed against PBS at 4° C. and centrifuged to remove any precipitated protein.

Hevein peptide: The 43 aa hevein peptide was synthesised according to the prohevein sequence of Rozynek et al. 1998, (supra) by Mimotopes (Clayton, Australia).

Control Allergens: House Dust Mite extract (HDM), KLH

Site-Directed Mutagenesis of rHev b 6

Site directed mutagenesis of the pPROEX-HTa/Hev b 6 construct was carried out using the QuickChange™XL site direct mutagenesis kit (Stratagene, CA, USA) according to the manufacturer's instructions. The primers B6C1AF (5′-GAA TTC GAG CAA GCA GGT CGG CAA GCA GGT GGC AAG C-3′) (SEQ ID NO:24) and B6C1AR (5′-GCT TGC CAC CTG CTT GCC GAC CTG CTT GCT CGA ATT C-3′) (SEQ ID NO:25) were used to replace the cysteine at amino acid three of the mature protein with an alanine. After site-directed mutagenesis the construct was sequenced to confirm the nucleotide changes. The construct containing the mutant Hev b 6 was transformed into E. coli strain BL21-CodonPlus® (DE3)-RIL competent cells, expressed and purified as above. Mutant rHev b 6 was refolded as for rHev b 6 (see above).

ELISA for Latex Specific Human IgE and Hev b 6-Specific mAb

Patient serum samples were tested for Hev b 6 specific IgE using our standard ELISA protocol (Sutherland et al. 2002, supra) with rabbit anti-human IgE (Dako, Denmark) at 1 in 1000 dilution followed by swine anti-rabbit IgG-HRP conjugate (Promega) at I in 1000 dilution. For the Hev b 6-specific mAb 1A5.4 (IgG1/kappa isotype) binding was detected using sheep anti-mouse Ig-HRP (Silenus) at 1 in 1000 dilution.

Gel Electrophoresis and Western Blotting

Four μg of rHev b 6 or sulphonated rHev b 6 were separated on 16% sodium dodecyl sulphate (SDS) polyacrylamide gels with 4% stacking gel. Electrophoresis was performed at 125V for 2 h on a Novex Xcell II Mini-Cell system (Novex, CA, USA). Proteins were visualized with Coomassie Brilliant Blue R-250 (Sigma). The separated proteins were electrophoretically transferred to nitro-cellulose membranes (BA 0.45μ, Schleicher and Schuell, Dassel, Germany) at 25V for 1 hr in transfer buffer. Membranes were blocked for 1 h at RT in 10% skim milk powder (SMP) in PBS, washed once in 0.05% Tween20/PBS and twice in PBS. Washed membranes were incubated for 1 hour at RT with either culture supernatant containing the Hev b 6-specific mAb 1A5.4, diluted 1 in 100 in 1% SMP/PBS or human serum, diluted 1 in 5 in 1% SMP/PBS. For mAb detection membranes were washed as above and incubated for 1 h at RT with goat HRP-conjugated anti-mouse Ig (Silenus), diluted 1 in 2000 in 1% SMP/PBS. For IgE detection, membranes were incubated for 1 h at RT with rabbit anti-human IgE (Dako, Denmark) at 1 in 1000 dilution, followed by swine anti-rabbit IgG-HRP conjugate (Promega) at 1 in 1000 dilution. After washing, the binding of 1A5.4 mAb and patient serum IgE to rHev b 6 was detected using hydrogen peroxide/diaminobenzidine solution.

Generation of Latex Specific T Cell Lines

PBMC were collected from heparinized venous blood by density gradient centrifugation on Ficoll Paque (Pharmacia Biotech, USA) and Hev b 6 specific T-cell lines were generated using established methods (de Silva et al., 2000, supra; O'Hehir et al., 1987, supra; O'Hehir R E et al., 1993; Eur J Clin Invest., 23:763-772). PBMC were stimulated with GE at 20 μg/ml, in 24-well tissue culture plates (Costar, USA) at a density of 2.5×10⁶ PBMC/well in complete medium (RPMI-1640 supplemented with 2 mM L-glutamine, 100 IU/ml penicillin-streptomycin [Invitrogen, USA] and 5% screened, heat inactivated human AB serum [Sigma, USA]). After 7 days, lymphoblasts were restimulated (1×10⁶ cells/well) for a further week with 20 μg/ml GE in the presence of equal number of irradiated (3000 Rad) autologous PBMC as APC. On days 2 and 4, 5% Lymphocult T (Biotest Folex, Germany) was added to the cultures. After 2 weeks, lymphoblasts were stimulated as above with 20 μg/ml sulphonated rHev b 6 to enrich for Hev b 6-specific T-cells. After 3 weeks, resting oligoclonal T-cell blasts were recovered, washed and tested in proliferation assays as described below. We have previously shown that CD4+ T cells are preferentially expanded in these cultures (O'Hehir et al., 1987, supra).

Oligoclonal T-Cell Proliferation Assays

Oligoclonal T-cell blasts (5×10⁴/well) from the 3-week cultures were stimulated with GE, hevein peptide, refolded rHev b 6 and refolded mutant rHev b 6 over a range of concentrations (2.5 to 10 μg/ml), in the presence of equal numbers of APC. Wells containing T cells and APC in the absence of Ag were included to assess background levels of cell proliferation. After 72 h in culture proliferation was determined by pulsing with 1 μCi of ³HTdR (Du Pont) for 12 to 16 h and liquid scintillation spectroscopy. The results were expressed as mean cpm for triplicate cultures. Stimulation indices (SI; cpm antigen stimulated cultures divided by cpm of unstimulated T cells) were calculated with SI≧2.5 defined as positive. Mitogenicity and toxicity of all latex allergens were excluded (data not shown).

Basophil Activation Test

A basophil activation assay based on flow cytometry was used to quantify the activation of basophils after incubation with various allergens. This assay was modified from Paris-Kohler A, Demoly P, Persi L, Lebel B, Bousquet J, Arnoux B. In vitro diagnosis of cypress pollen allergy by using cytofluorimetric analysis of basophils (Basotest). J Allergy Clin Immunol 2000; 105:339-45. Whole blood was collected in 6 ml heparinised vacutainers (Greiner). 100 μl aliquots were preincubated with stimulation buffer (20 mM Hepes, 133 mM NaCl, 5 mM KCl, 7 mM CaCl₂, 3.5 mM MgCl₂, 1 mg/ml BSA, 20 μl/ml heparin, pH 7.4) containing 2 ng/ml IL-3, for 10 min at 37° C., to increase the sensitivity of the assay. Samples were incubated for 20 min at 37° C. with 100 μl of stimulation buffer alone or stimulation buffer containing either rabbit anti-human IgE (Dako) at 1 in 500 dilution, 2 μM N-fMLP (Sigma, Mo, USA), crude allergen extracts, recombinant latex allergens or Keyhole Limpet Haemocyanin (KLH) (Sigma, Mo, USA). Basophil activation was stopped by incubating at 4° C. for 5 min. Normal goat serum (10 μl) was added to each tube to block non-specific binding of the detection antibody (goat anti-human IgE-FITC) and incubated for 10 min at 4° C. Samples were incubated with goat anti-human IgE-FITC (Caltag, Birmingham, Calif.) at a dilution of 1 in 2.5, and mouse anti-human CD63-R-Phycoerythrin (PE; Caltag, Birmingham, Calif.) at a dilution of 1 in 20, in a 20 μl volume of wash buffer (20 mM HEPES, 133 mM NaCl, 5 mM KCl, 0.27 mM EDTA, pH7.3). RBC were lysed by the addition of 2 ml FACS lysing solution (39 mM NH₄Cl, 2.5 mM KHCO₃, 0.2 mM EDTA) and incubation for 10 min at room temperature. Cells were centrifuged (250 g, 5 min at 4° C.), resuspended in 3 ml wash buffer, and centrifuged as before. Pellets were resuspended in 200 μl wash buffer, and analysed by flow cytometry (FACScalibur, Becton Dickinson) within 2 h of the experiment. 7AAD (Sigma, Mo, USA) was added to the samples 5 min prior to FACS analysis.

### EXAMPLE 8

### Chemical Disruption of the Disulphide Bonds of rHev b 6 Abolishes IgE Reactivity

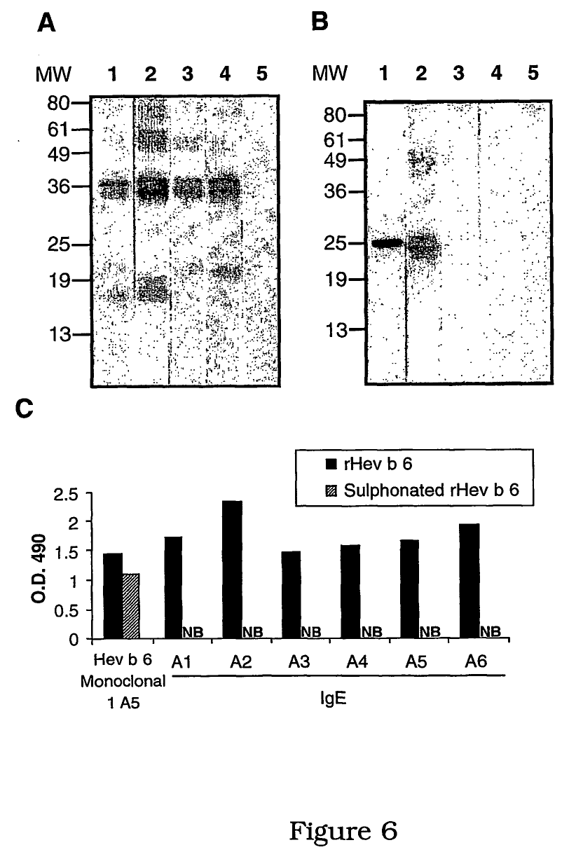

rHev b 6 expressed in bacteria with a 6-His tag was insoluble and was purified under denaturing conditions with 8M urea to solubilize the protein. On SDS-PAGE the purified recombinant protein appeared as multiple bands under non-reducing conditions (FIG. 6A, lane 1). When Western blotting was carried out these bands were reactive with both a Hev b 6-specific mAb (FIG. 6A, lane 2) and IgE from latex allergic subjects (FIG. 6A, lanes 3 and 4). A control serum from a non-latex allergic subject did not react with rHev b 6 (FIG. 6A, lane 5). Under reducing conditions on SDS-PAGE the multiple bands collapsed to one major band, at the expected molecular mass of 20 kDa and one minor band at 40 kDa. Both bands were reactive with the mAb and IgE (data not shown). The multiple bands seen under non-reducing conditions appeared to represent dimerization of rHev b 6, presumably due to intermolecular disulphide bonding.

The breaking of disulphide bonds, and the subsequent capping of the cysteine residues, can increase the solubility of recombinant proteins (De Bernardez Clark, 1998, supra). The solubility of rHev b 6 was increased markedly after sulphonation. On SDS-PAGE, under non-reducing conditions, sulphonated rHev b 6 ran as a single band at an apparent molecular mass of 25 kDa (FIG. 6B, lane 1). This band was reactive with the Hev b 6-specific mAb (FIG. 6B, lane 2) but failed to react with IgE from patient sera (FIG. 6B, lanes 3 to 5). Sulphonation is reversible under reducing conditions (De Bernardez Clark, 1998, supra) thus as expected under reducing conditions on SDS-PAGE sulphonated rHev b 6 ran at a molecular mass of around 20 kDa with a second minor band at 40 kDa (data not shown), as seen for the non-sulphonated rHev b 6. Again both these bands were reactive with the mAb and IgE (data not shown).

To confirm the loss of IgE reactivity of rHev b 6 after sulphonation, the ability of both the mAb and IgE from patient sera to bind non-sulphonated and sulphonated rHev b was determined by ELISA. Both forms of rHev b 6 were bound to a similar extent by the mAb (FIG. 6C). However, IgE from the sera of 7 latex allergic, Hev b 6 sensitised subjects bound only the non-sulphonated form of rHev b 6, with no detectable binding of IgE to sulphonated rHev b 6.

### EXAMPLE 9

### Disruption of the First Disulphide Bond of Hevein Abolishes IgE Reactivity of rHev b 6

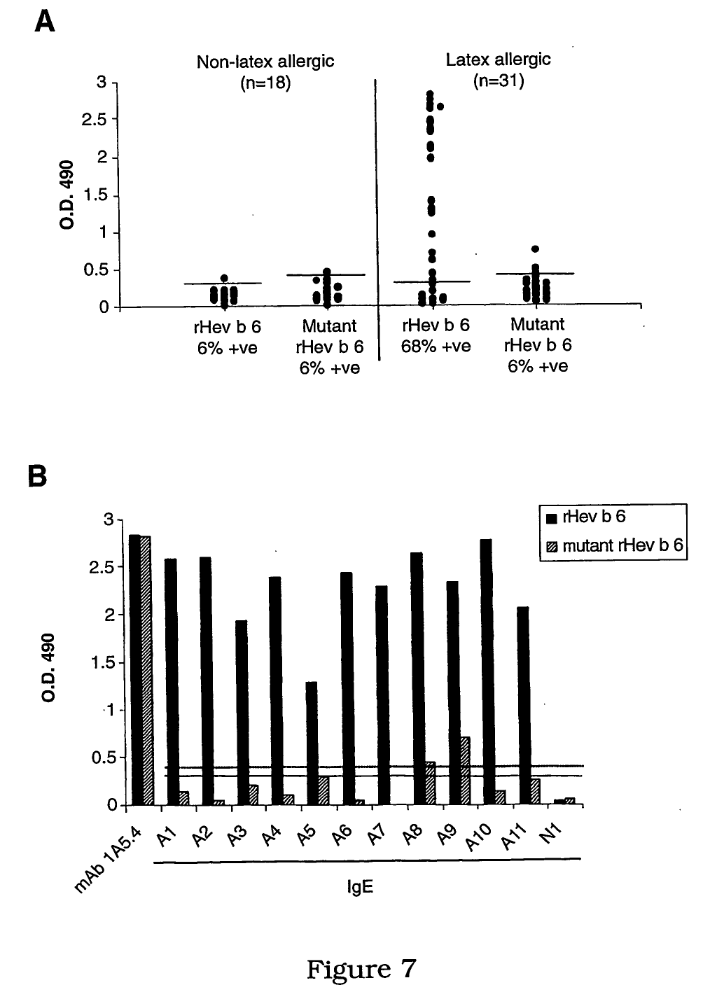

To further investigate the contribution of the disulphide bonds of Hev b 6 to its IgE reactivity, an alanine was substituted for a cysteine at amino acid 3 of the hevein domain. This cysteine pairs with another at amino acid 31 forming the first disulphide bond of Hev b 6. To ascertain whether this substitution affected the ability of IgE to bind rHev b 6, IgE binding to rHev b 6 and mutant rHev b 6 was assayed by ELISA (FIG. 7A). Thirty-one latex allergic and 18 non-latex allergic subjects were assayed. The mean plus two SD of the ELISA scores of the latex non-allergic subjects was used as a cut off for a positive reading. In keeping with other studies (Yip L, Hickey V, Wagner B, Liss G, Slater J, Breiteneder H, Sussman G, Beezhold D. Skin prick test reactivity to recombinant latex allergens. Int Arch Allergy Immunol 2000; 121:292-299; Banerjee B, Wang X, Kelly K J, Fink J N, Sussman G L and Kurup V P. IgE from latex-allergic patients binds to cloned and expressed B cell epitopes of prohevein. J Immunol 1997; 159:5724-32), 68% of latex allergic subjects were positive to rHev b 6 (21/31; FIG. 7A). In contrast IgE from only 6% of latex allergic subjects (2/31) bound mutant Hev b 6. This was the same frequency of reactivity found with the non-latex allergic subjects for both rHev b 6 and mutant rHev b 6. With the two latex allergic subjects who had positive IgE binding to mutant rHev b 6, IgE binding was reduced by between 60 and 80% compared with rHev b 6 (FIG. 7B, subjects A8 and A9). When the Hev b 6-specific mAb was used in the same assay, it retained comparable reactivity with rHevb 6 and mutant rHev b 6 (FIG. 7B). The results of the ELISA demonstrate that disruption of the first disulphide bond of Hev b 6 abrogated or markedly decreased IgE binding to this protein.

### EXAMPLE 10

### Mutant rHev b 6 is a Poor Inhibitor of IgE Binding to rHev b 6

To confirm the lack of IgE reactivity of mutant rHev b 6, inhibition ELISA were performed. In these assays, IgE binding to rHev b 6 was inhibited by preincubation of sera from latex allergic, Hev b 6 reactive subjects with either rHev b 6 or mutant rHev b 6 over a range of concentrations. To determine the level of non-specific inhibition inherent in such an assay, an initial non-specific inhibition assay was performed. The serum of a latex-allergic, Hev b 5 positive subject was incubated with rHev b 6 and mutant rHev b 6 and then the ability of IgE from this serum to bind rHev b 5 in an ELISA was measured. When this assay was undertaken, binding of IgE to rHev b5 was inhibited at a maximum of 20% by rHev b 6 and mutant rHev b 6 (data not shown). Given these results the level for positive inhibition was set at greater than 20% for rHev b 6 and mutant rHev b 6.

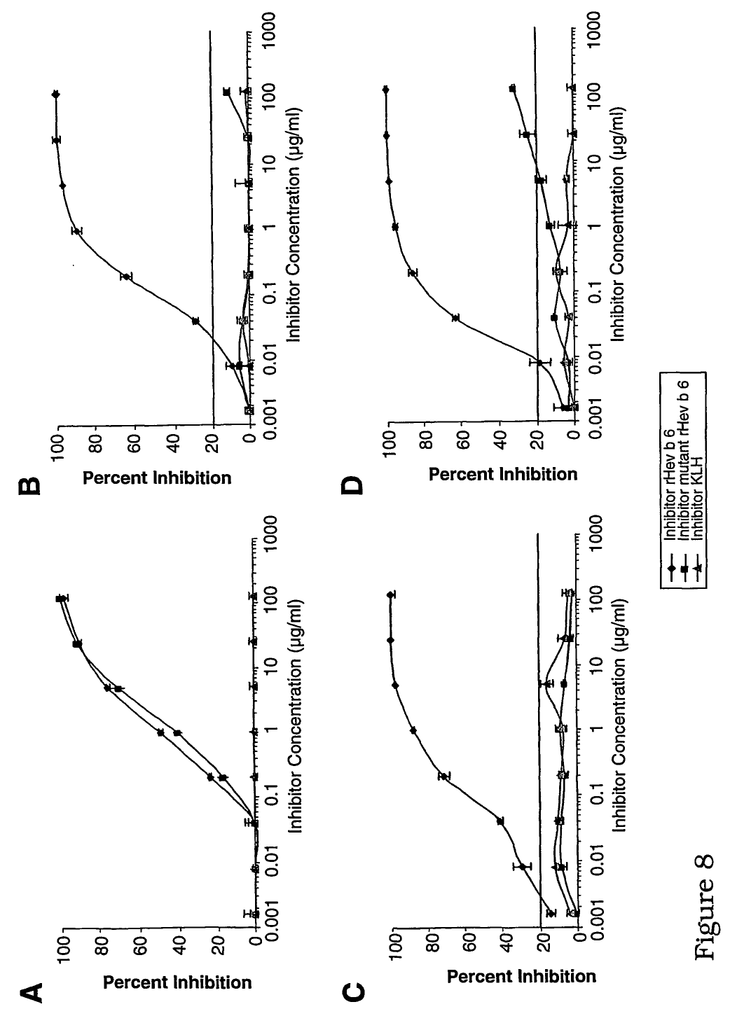

When rHev b 6 and mutant rHev b 6 were used to inhibit the binding of IgE to rHev b 6 (FIGS. 8B, C and D), as expected rHev b 6 produced positive inhibition at low inhibitor concentrations, increasing to 100% at higher concentrations. In contrast, mutant rHev b 6 failed to inhibit above the level determined for non-specific inhibition for two subjects (FIGS. 8B and C). For a third subject the mutant rHev b 6 inhibited above the cut-off for non-specific inhibition at the two highest concentrations of inhibitor (FIG. 8D). The maximum inhibition obtained with mutant rHev b 6 for this subject was 32% at 125 μg/ml, compared to the rHev b 6, which reached 100% inhibition at this inhibitor concentration.

In order to confirm the ability of the mutant rHev b 6 to inhibit Ab binding, rHev b 6 and mutant rHev b 6 were used to inhibit binding of the Hev b 6-specific mAb 1A5.4 to rHev b 6 (FIG. 8A). Both rHev b 6 and mutant rHev b 6 showed comparable inhibition of mAb binding to rHev b 6.

To compare the relative ability of IgE to bind rHev b 6 and mutant rHev b 6, the inhibitor concentrations required to give 30% inhibition of IgE binding were estimated. For rHev b 6, 30% inhibition was reached at 0.01 μg/ml and for mutant rHev b 6, 30% inhibition was reached at 125 μg/ml. Therefore, 12,500 fold more mutant rHev b 6 was required to inhibit IgE binding as effectively as rHev b 6.

### EXAMPLE 11

### Mutant rHev b 6 has Decreased Basophil Activation Compared with rHev b 6

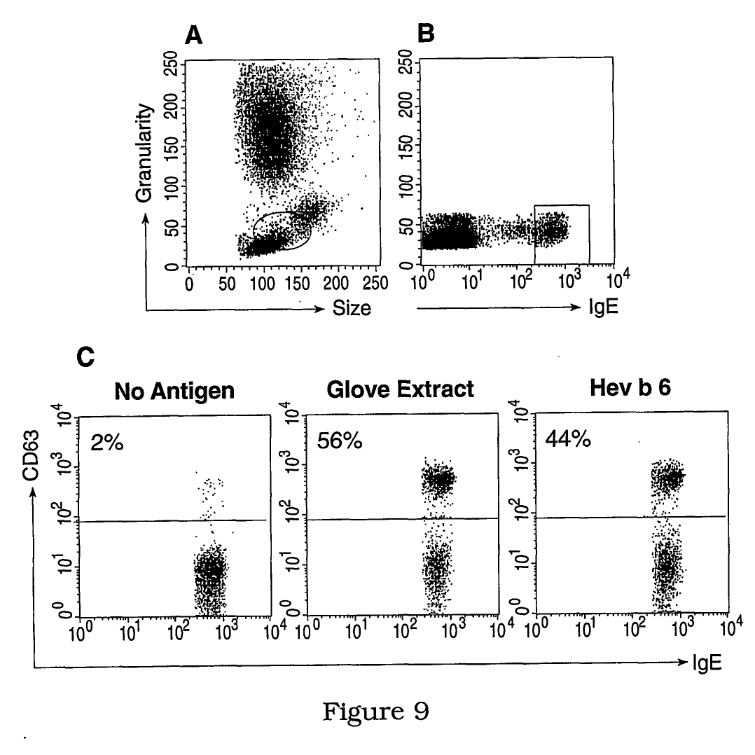

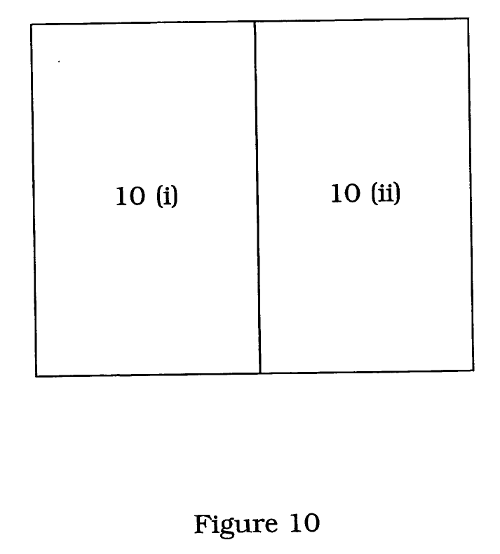

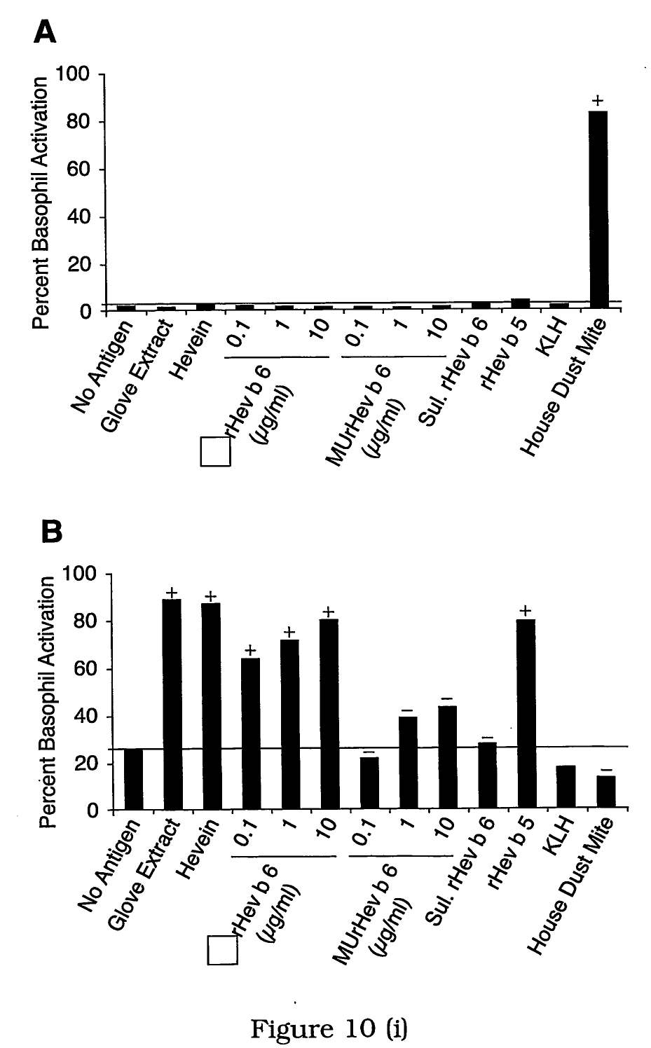

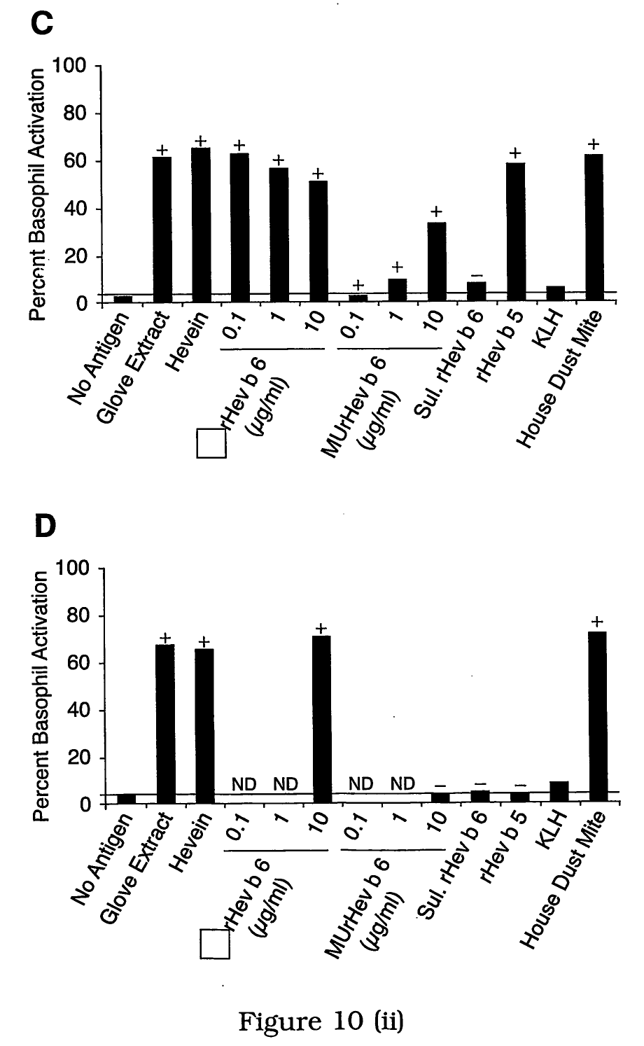

To further investigate the biological potency of the mutant rHevb 6, the ability of mutant rHev b 6 and rHev b 6 to activate basophils was assessed using the Basophil Activation Test (BAT; Paris-Kohler et al., 2001, supra). Sample BAT flow cytometry plots are shown in FIG. 4 for a latex allergic, Hev b 6 sensitized subject. These plots demonstrate the marked increase in CD63 positive high IgE staining cells seen after stimulation with allergen. The results of BAT for three latex allergic, Hev b 6 sensitised subjects (A2, A7, A10) and a non-latex allergic subject (N2) are shown (FIG. 10). When the control subject's basophils were stimulated with GE, the hevein peptide, rHev b 5, KLH (as a negative control antigen), rHev b 6 and mutant rHev b 6 at various concentrations, negligible basophil activation was evident (FIG. 9A). In contrast when the control subject's basophils were stimulated with the relevant aeroallergen extract, HDM (Table 7; FIG. 9D), CD63 positive staining was evident in 80% of high IgE staining cells.

When basophils from latex allergic, Hev b 6 sensitised subjects were stimulated with GE and the hevein peptide, between 70 and 90% of high IgE staining cells were CD63 positive (FIGS. 9B, C and D). Similar levels of basophil activation were observed when cells were stimulated with rHev b 6 at 0.1, 1 and 10 μg/ml. However, in comparison basophils stimulated with mutant rHev b 6 gave markedly decreased activation. At 0.1 μg/ml no activation above background levels (stimulation buffer alone) was seen with mutant rHev b 6 (FIGS. 9B and C), compared with 30-50% above background levels for rHev b 6. At Ag concentration of 1 μg/ml, activation levels increased to 5-10% over background for mutant rHev b 6 compared with 50-60% for the rHev b 6. At the highest concentration used, 10 μg/ml, activation levels remained low for one subject (FIG. 9B), 15% above background for mutant rHev b 6, compared to 60% above background for rHev b 6. For another patient (FIG. 9C), less of a difference was evident in the ability of mutant rHev b 6 and rHev b 6 to activate basophils at 10 μg/ml, with mutant rHev b 6 producing 40% activated basophils and rHev b 6, 60%. The third patient showed no activation of basophils in response to stimulation with mutant rHev b 6 at 10 μg/ml while 70% of basophils were activated by rHev b 6 at this concentration.

Interestingly sulphonated rHev b 6 (FIGS. 9B, C and D) was even less effective than the mutant rHev b 6 at activating basophils. Sulphonated rHev b 6 activated less than 5% of basophils (above background) at 10 μg/ml, compared to up to 49% for mutant rHev b 6 and 50 to 70% for rHev b 6.

### EXAMPLE 12

### Mutant rHev b 6 Retains the Ability to Stimulate Proliferation of Allergen-Specific T-Cells

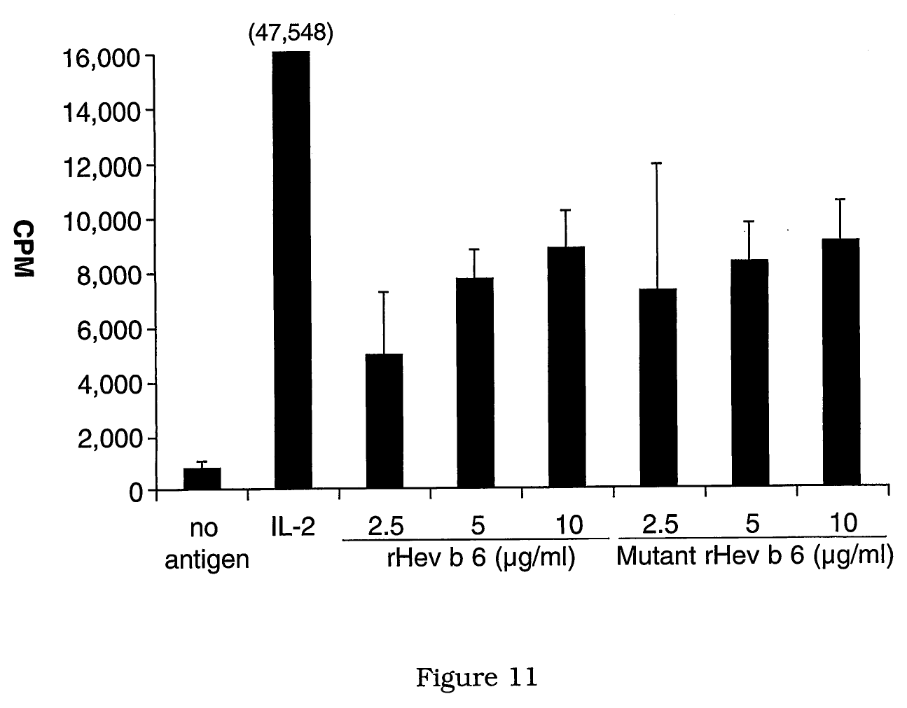

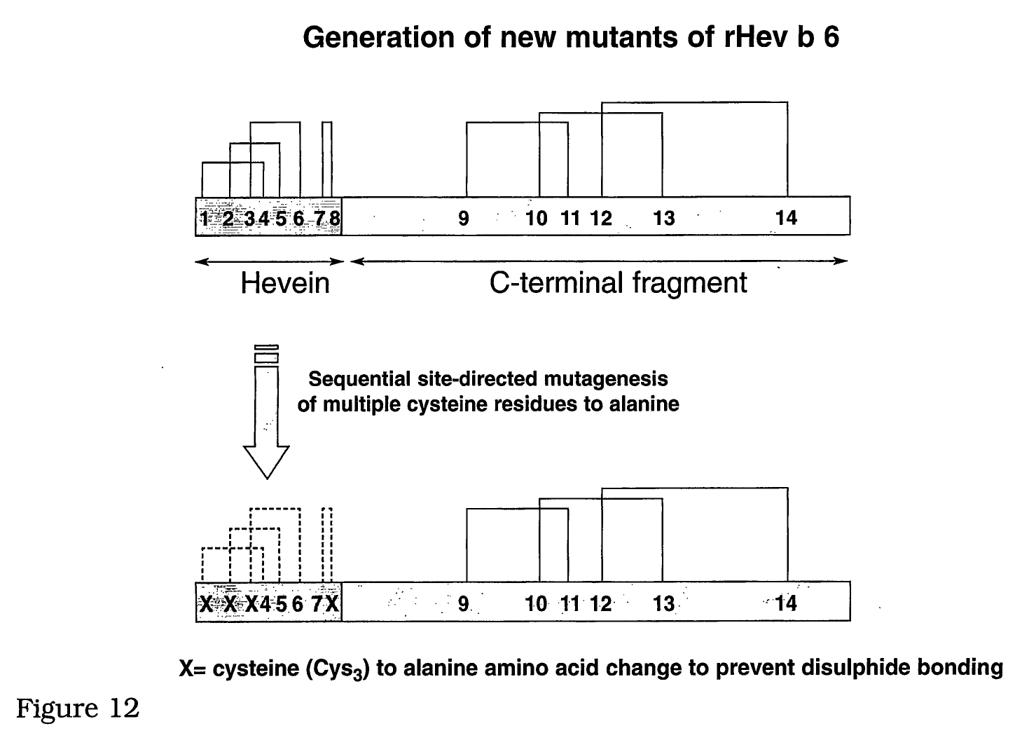

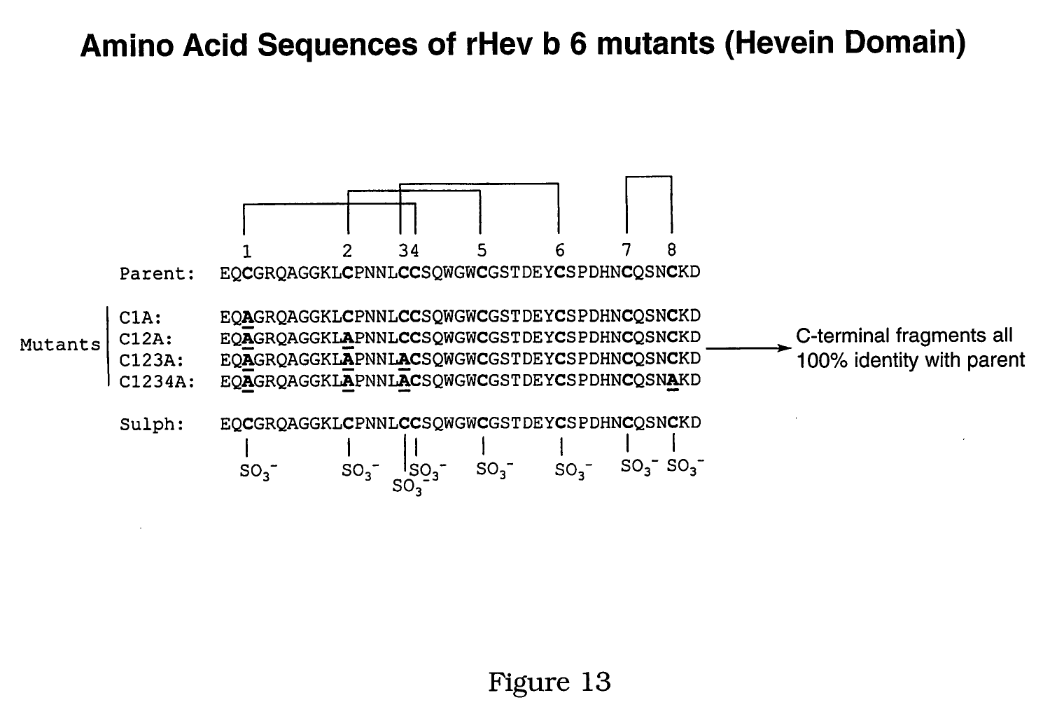

In order to determine whether mutant rHev b 6 retained T cell stimulatory activity, oligoclonal Hev b 6 specific CD4⁺ T cell lines were generated from six latex allergic Hev b 6 sensitised subjects (A1, A3, A4, A5, A8 and A11; Table 7). A representative proliferation assay is shown (FIG. 11). Oligoclonal T cell blasts from all subjects proliferated to both rHev b 6 and mutant rHev b 6, with SI≧2.5 in all cases (Table 8). The proliferation of Hev b 6-specific T cell lines to mutant rHev b 6 strongly suggests that the critical T-cell epitopes of Hev b 6 remain intact in mutant rHev b 6.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 1
Taxanomic classification of fruits, vegetables and other plant tissues reported to cross-react. References are cited by numbers in square
brackets following common names.

| | Sub- | Super- | | | | | | Genus, species |
|---|---|---|---|---|---|---|---|---|
| Kingdom | kingdom | division | Division | Class | Sub-class | Order | Family | (Common name) |
| Plantae | Tracheo- | Sperma- | Magnolio- | Magno- | Rosidae | Apiales | Apiaceac | Apium L. (Celery) |
| (Plants) | bionta | tophyta | phyta | liopsida | | | (Carrot family) | |
| | (Vascular | (Seed | (Flowering | (Dicotyl- | | Euphorbiales | Euphorbiaceae | Hevea brasiliensis |
| | plants) | plants) | plants) | edons) | | | (Spurge family) | (Wilid. Ex Adr. Juss.) |
| | | | | | | | | Muell. Arg. |
| | | | | | | | | (Rubber tree) |
| | | | | | | | | Mercurialis annua L. |
| | | | | | | | | (Annual mercury) |
| | | | | | | Rosales | Rosaceae | Malnus P. Mill (Apple) |
| | | | | | | | (Rose family) | Prunus (L.) (Plum) |
| | | | | | | | | Prunus avium (L.) |
| | | | | | | | | L. (Sweet cherry) |
| | | | | | | | | Prunus persica |
| | | | | | | | | (L.) Batsch (Peach) |
| | | | | | | | | Prunus persica |
| | | | | | | | | (L.) Batsch var. |
| | | | | | | | | Nucipersica (Suckow) C |
| | | | | | | | | Schneider (Nectarine) |
| | | | | | | Sapindales | Anacardiaceae | Mangifera indica L. |
| | | | | | | | (Sumac family) | (Mango) |
| | | | | | Magnoliidae | Lauraceae | Lauraceae | Persea americana |
| | | | | | | | (Laurel family) | P. Mill. (Avocado) |
| | | | | | | Magnoliales | Annonaceae | Annona cheirimola |
| | | | | | | | | P. Mill. (Cherimoya) |
| | | | | | | | (Custard-apple | Asimina triloba L. (Pawpaw) |
| | | | | | | | family) | |
| | | | | | Hamamelidae | Fagales | Fagaceae | Castanea sativa P. Mill. |
| | | | | | | | (Beech family) | (European chestnut) |
| | | | | | | Urticales | Moraceae | Artocarpus altilis (Parkinson) |
| | | | | | | | (Mulberry family) | Fosberg (Breadfruit) |
| | | | | | | | | Ficus benjamina |
| | | | | | | | | L. (Weeping fig) |
| | | | | | Dilleniidae | Ebenales | Ebenaceae | Diospyros virginiana L. |
| | | | | | | | (Ebony family) | (Persimmon) |
| | | | | | | Theales | Actinidiaceae | Actinidia chinensis |
| | | | | | | | (Chinese gooseberry | Planchon |
| | | | | | | | family) | (Kiwi fruit) |
| | | | | | | Violales | Cucurbitaceae | Cucumis L. (Melon) |
| | | | | | | | (Cucumber family) | Cucurbita pepo L. (Zucchini) |
| | | | | | Caryophyllidae | Polygonales | Polygonaceae | Eriogonm Michx. |
| | | | | | | | (Buckwheat family) | (Buckwheat) |
| | | | | | Asteridae | Asterales | Asteraceae | Lactuca sativa L. |
| | | | | | | | (Aster family) | (Lettuce) |
| | | | | | | Solanales | Solanaceae | Solanum lycopersicum |
| | | | | | | | | L. (Tomato) |
| | | | | | | | (Potato family) | Solanum tuberosum |
| | | | | | | | | L. (Potato) |
| | | | | Liliopsida | Zingiberidae | Bromeliales | Bromeliaceae | Ananas comosus (L.) Merr. |
| | | | | (Monocotyledons) | | | (Bromeliad family) | (Pineapple) |
| | | | | | | Zingiberales | Musaceae | Musa acuminata Colla |
| | | | | | | | (Banana family) | (Banana) |
| | | | | | Commelinidae | Cyperales | Poaceae | Pleum pratense L. |
| | | | | | | | (Grass family) | (Timothy grass) |

### BIBLIOGRAPHY

Abeck D et al., 1994; Hautarzt., 45(6):364-7.
Akasawa A et al., 1996; J Biol. Chem., 271:25389-25393.
Alenius H et al., 1996; Clin Exp Allergy., 26(3):341-8.
Anliker M D et al., 2001; J Allergy Clin Immunol., 107(4):718-23.
Antico A., 1996; Ann Allergy Asthma Immunol., 76(1):37-40.
Banerjee B, Wang X, Kelly K J, Fink J N, Sussman G L and Kurup V P. IgE from latex-allergic patients binds to cloned and expressed B cell epitopes of prohevein. J Immunol 1997; 159:5724-32
Blanco C et al., 1994; Ann Allergy., 73(4):309-14.
Blanco C et al., 1994; Allergy., 49(6):454-9.
Blanco C et al., 1999; J Allergy Clin Immunol., 103(3 Pt 1):507-13.
Broekaert W, Lee H, Kush A, Chua N-H, Raikhel N. Wound-induced accumulation of mRNA containing a hevein sequence in laticifers of rubber tree (Hevea brasiliensis). Proc Natl Acad Sci USA 1990; 87:7633-7637
Brown R H et al., 1998; Anaesthesiology., 89:292-299.
Brugnami G et al., 1995; J Allergy Clin Immunol., 96:457-464.
Chen Z et al., 1996; Clin Exp Allergy., 26:406-415.
Chen Z et al., 1997; J Allergy Clin Immunol., 99(3):402-9.
Chen Z et al., 1998; J Allergy Clin Immunol., 102:476-481.
De Bernardez Clark E R. Refolding of recombinant proteins. Curr Opin Biotechnol 1997; 9:157-163
De Greef J M et al., 2001; Int Arch Allergy Immunol., 125(2):182-4.
De Silva H D, Sutherland M F, Suphioglu C, McLellan S C, Slater J E, Rolland J M, O'Hehir R E. Human T-cell epitopes of the latex allergen Hev b 5 in health care workers. J Allergy Clin Immunol 2000, 105:1017-1024
Douglas R et al., 1997; Aust NZ J Med., 27:165-169.
Duque S et al., 1999; Allergy., 54(9):1004-5.
Garcia Ortiz J C et al., 1998; Allergy., 53(5):532-6.
Goding, J. Monoclonal Antibodies: principles and practice 3rd ed. London: Academic Press. 492, 1996.
Grzybowski M et al., 1996; J. Allergy Clin Immunol., 98:535-544.
Jameel et al., 1990; J. Virol., 64:3963-3966.
Kibby T and Akl M., 1997; Ann Allergy Asthma Immunol., 78:41-44.
Knapp et al., 1990; Bio Techniques., 8:280-281.
Köhler G. and Milstein C., 1975; Nature., 256(5517):495-7.
Kostyal D A et al., 1998; Clin Exp Immunol., 112:355-362.
Kurjan and Herskowitz., 1982; Cell., 30:933-943.
Levy D A et al., 2000; Clin Exp Allergy., 30(2):270-5.
Liss G M et al., 1997; Occ Environ Med., 54:335-342.
Mikkola J H et al., 1998; J Allergy Clin Immunol., 102(6 Pt 1): 1005-12.
O'Hehir R E et al., 1987; Immunology., 62:635-640.
O'Hehir R E et al., 1993; Eur J Clin Invest., 23:763-772.
O'Hehir R E, Askonas B A and Lamb J R., 1993, Cell culture: lymphocyte clones. In: Albert W H W, Staines N A editors. Methods of immunological analysis. Vol 3. Heideliberg: Springer-Verlag; 1993: p120-38.
Ownby D R et al., 1991; AJR., 156:903-908.
Paris-Kohler A, Demoly P, Persi L, Lebel B, Bousquet J, Arnoux B. In vitro diagnosis of cypress pollen allergy by using cytofluorimetric analysis of basophils (Basotest). J Allergy Clin Immunol 2000; 105:339-45
Posch A et al., 1998; Clin Exp Allergy., 28:134-140.
Raulf-Heimsoth M et al., 1998; Clin Exp Allergy., 28:339-348.
Rozynek P et al., 1998, Clinical and Experimental Allergy 28:1418-1426.
Sanchez-Guerrero I M et al., 2000; Allergy., 55(10):976-7.
Sambruck et al; “Molecular cloning: a laboratory manual, Second Edition”; Cold Spring Harbour Laboratory Press; Cold Spring Harbour, N.Y.; 1989.
Scheiner O et al., 1999; Int Arch Allergy Immunol., 118:311-312.
Schultz et al., 1987; Gene., 54:113-123.
Seppala U et al., 2000; Allergy., 55(3):266-73.
Slater J E et al., 1996; J Biol. Chem., 271:25349-24399.
Slater J E., 1994; J Allergy Clin Immunol., 94:139-149.
Slater, J. E et al., 1996; Journal of Biological Chemistry., 271(41):25389-93.
Sowka S et al., 1998; Eur J Bioch., 255:213-219.
Suphioglu C et al., 1993; Allergy., 48:273-281.
Sussman G L et al., 1991; JAMA., 265:2844-2847.
Sutherland M F, Drew A, Rolland J M, Slater J E, Suphioglu C, O'Hehir R E. Specific monoclonal antibodies and human immunoglobulin E show that Hev b 5 is an abundant allergen in high protein powdered latex gloves. Clin Exp Allergy 2002; 32:583-589
Varney et al., 1990., British Medical Journal., 302:265-269.
Weiss S J et al., 1996., Ann Allergy Asthma Immunol., 77(6):504-8.
Yeang H. Y. et al., 1996., J Allergy Clin Immunol; 98:628-639.
Yip L, Hickey V, Wagner B, Liss G, Slater J, Breiteneder H, Sussman G, Beezhold D. Skin prick test reactivity to recombinant latex allergens. Int Arch Allergy Immunol 2000; 121:292-299

1. An isolated peptide comprising a Hev b 6 T cell epitope said peptide comprising at least 5 continuous amino acids of an amino acid sequence selected from the group consisting of:
(i) amino acids 1-47;
(ii) amino acids 55-83;
(iii) amino acids 82-101;
(iv) amino acids 136-155;
inclusive, of Hev b 6 (e.g. SEQ ID NO:1);
and wherein said peptide molecule is capable of interacting with T cells and modifying T cell function when incubated with cells from subjects having a condition characterized by an aberrant, unwanted or otherwise inappropriate immune response to Hev b 6.

2. (canceled)

3. The isolated peptide according to claim 1 wherein said amino acid sequence is amino acids 1-20, inclusive of Hev b 6.

4. (canceled)

5. The isolated peptide according to claim 1 wherein said amino acid sequence is amino acids 10-29 inclusive of Hev b 6.

6. The isolated peptide according to claim 1 wherein said amino acid sequence is amino acids 19-38 inclusive of Hev b 6.

7. The isolated peptide according to claim 1 wherein said amino acid sequence comprises at least 5 consecutive amino acids from an amino acid sequences selected from the group consisting of:

| | |
|---|---|
| EQCGRQAGGKLCPNNLCCSQ | (SEQ ID NO:2) |
| KLCPNNLCCSQWGWCGSTDE | (SEQ ID NO:3) |
| SQWGWCGSTDEYCSPDHNCQ | (SEQ ID NO:4) |
| and | |
| DEYCSPDHNCQSNCKDSGEG. | (SEQ ID NO:5) |

8. The peptide according to claim 7 wherein said amino acid sequence is from SEQ ID NO: 3.

9. The peptide according to claim 7 wherein said amino acid sequence is from SEQ ID NO: 4.

10. The peptide according to claim 1 wherein said peptide exhibits reduced or ablated IgE binding relative to Hev b 6 having the amino acid sequence of SEQ ID NO:1.

11. (canceled)

12. The peptide according claim 10 wherein disulphide bond formation is abrogated relative to Hev b 6 having the amino acid sequence of SEQ ID NO:1.

13. The peptide according to claim 12 wherein the disulphide bond formation that is abrogated involves a cysteine residue at an amino acid position selected from the group consisting of: 3.12, 17, 18, 24, 31, 37 and 41 of SEQ ID NO: 1.

14. The peptide according to claim 13 wherein said cysteine amino acid at position 3 or 8 is substituted with an alanine amino acid.

15-28. (canceled)

29. An antibody directed to a Hev b 6 T cell epitope, said epitope being a peptide comprising at least 5 contiguous amino acids of an amino acid sequence selected from the group consisting of:
(i) amino acids 1-47;
(ii) amino acids 55-83;
(iii) amino acids 82-101;
(iv) amino acids 136-155;
inclusive, of Hev b 6 (e.g. SEQ ID NO: 1);
wherein said peptide molecule is capable of interacting with T cells and modifying T cell function when incubated with cells from subjects having a condition characterized by an aberrant, unwanted or otherwise inappropriate immune response to Hev b 6.

30. The antibody according to claim 29 wherein said antibody is a polyclonal antibody.

31. The antibody according to claim 29 wherein said antibody is a monoclonal antibody.

32. The antibody according to claim 31 wherein said antibody is produced by hybridoma 1A5.4 (ECACC Accession No. 01122118) or 6E5.3.

33. The hybridoma 1A5.4 (ECACC Accession No. 01122118) or 6E5.3.

34-37. (canceled)

38. An isolated nucleic acid sequence encoding or complementary to a sequence encoding a Hev b 6 T cell epitope, said epitope being a peptide comprising at least 5 contiguous amino acids of an amino acid sequence selected from the group consisting of:
(i) amino acids 1-47;
(ii) amino acids 55-83;
(iii) amino acids 82-101;
(iv) amino acids 136-155;
inclusive, of Hev b 6 (e.g. SEQ ID NO:1);
wherein said peptide molecule is capable of interacting with T cells and modifying T cell function when incubated with cells from subjects having a condition characterized by an aberrant, unwanted or otherwise inappropriate immune response to Hev b 6

39. A method for the treatment and/or prophylaxis of a condition in a subject, which condition is characterised by an aberrant, unwanted or otherwise inappropriate immune response to Hev b 6 or functional homologue thereof, said method comprising administering to said subject an effective amount of a peptide according to claims 1, an antibody according to claim 29 or a nucleic acid molecule of claim 38 for a time and under conditions sufficient to remove or reduce the presence or function in said subject of T cells and/or antibodies directed to said Hev b 6.

40. The method according to claim 39 wherein said condition is latex hypersensitivity.

41. The method according to claim 39 wherein said condition is sensitivity to one or more fruits, vegetables and/or nuts which contain class 1 chitinases.

42. The method according to claim 41 wherein said fruit is a banana, avocado, or kiwi fruit and said nut is a chestnut.

43-46. (canceled)

47. A pharmaceutical composition comprising a peptide according to claims 1, an antibody according to claim 29 or a nucleic acid molecule of claim 38 and one or more pharmaceutically acceptable carriers and/or diluents.

48. A method of diagnosing or monitoring a condition in a mammal, which condition is characterised by an aberrant, unwanted or inappropriate response to Hev b 6, said method comprising screening for Hev b 6 reactive T cells and/or antibodies utilising the peptides according to claim 1.

49. The method according to claim 48 wherein said condition is latex hypersensitivity.

50. The method according to claim 48 wherein said condition is sensitivity to one or more fruits, vegetables and/or nuts which contain class 1 chitinases.

51. The method according to claim 50 wherein said fruit is banana, avocado, or kiwi fruit and said nut is a chestnut.

52. A method of qualitatively and/or quantitatively detecting Hev b 6, or peptides thereof, in a sample said method comprising screening for said Hev b 6 or peptides thereof based on their ability to bind to an antibody according to claim 29.

53. A diagnostic kit for diagnosis of a condition characterized by an aberrant, unwanted or otherwise inappropriate immune response to Hev b 6, said kit comprising a peptide according to claim 1 or an antibody according to claim 29.

54. The isolated peptide according to claim 1 wherein said amino acid sequence is amino acids 28-47 inclusive of Hev b 6.

55. The isolated peptide according to claim 1 wherein said amino acid sequence is amino acids 55-74 inclusive of Hev b 6.

56. The isolated peptide according to claim 1 wherein said amino acid sequence is amino acids 64-83 inclusive of Hev b 6.

57. The isolated peptide according to claim 1 wherein said amino acid sequence is amino acids 82-101 inclusive of Hev b 6.

58. The isolated peptide according to claim 1 wherein said amino acid sequence is amino acids 136-155 inclusive of Hev b 6.

59. The peptide according to claim 1 wherein said modification of T cell functioning is induction of T cell differentiation.

60. The peptide according to claim 13 wherein the cysteine amino acids at positions 3 and 12 and/or 18 and 24 are substituted with an alanine amino acid.

61. The peptide according to claim 13 wherein the cysteine amino acids at positions 3, 12 and 17 are substituted with an alanine amino acid.

62. The peptide according to claim 13 wherein the cysteine amino acids at positions 18, 24 and 31 are substituted with an alanine amino acid.

63. The peptide according to claim 13 wherein the cysteine amino acids at positions 3, 12, 17 and 41 are substituted with an alanine amino acid.

64. The peptide according to claim 13 wherein at least one of the cysteine amino acids at positions selected from the group consisting of 18, 24, 31 and 37 are substituted with an alanine amino acid.


SEQUENCE LISTING (31 sequences)

SEQ ID NO: 1 - protein, 187 aa, Hevea brasiliensis
EQCGRQAGGKLCPNNLCCSQWGWCGSTDEYCSPDHNCQSNCKDSGEGVGGGSASNVLATYHLYNSQDHGWDLNAASAYCSTWDANKPYSWRSKYGWTAFCGPVGAHGQSSCGKCLSVTNTGTGAKTTVRIVDQCSNGGLDLDVNVFRQLDTDGKGYERGHITVNYQFVDCGDSFNPLFSVMKSSVIN

SEQ ID NO: 2 - protein, 20 aa, Hevea brasiliensis
EQCGRQAGGKLCPNNLCCSQ

SEQ ID NO: 3 - protein, 20 aa, Hevea brasiliensis
KLCPNNLCCSQWGWCGSTDE

SEQ ID NO: 4 - protein, 20 aa, Hevea brasiliensis
SQWGWCGSTDEYCSPDHNCQ

SEQ ID NO: 5 - protein, 20 aa, Hevea brasiliensis
DEYCSPDHNCQSNCKDSGEG

SEQ ID NO: 6 - protein, 20 aa, Hevea brasiliensis
CQSNCKDSGEGVGGGSASNV

SEQ ID NO: 7 - protein, 20 aa, Hevea brasiliensis
EGVGGGSASNVLATYHLYNS

SEQ ID NO: 8 - protein, 20 aa, Hevea brasiliensis
NVLATYHLYNSQDHGWDLNA

SEQ ID NO: 9 - protein, 20 aa, Hevea brasiliensis
NSQDHGWDLNAASAYCSTWD

SEQ ID NO: 10 - protein, 20 aa, Hevea brasiliensis
NAASAYCSTWDANKPYSWRS

SEQ ID NO: 11 - protein, 20 aa, Hevea brasiliensis
WDANKPYSWRSKYGWTAFCG

SEQ ID NO: 12 - protein, 20 aa, Hevea brasiliensis
RSKYGWTAFCGPVGAHGQSS

SEQ ID NO: 13 - protein, 20 aa, Hevea brasiliensis
CGPVGAHGQSSCGKCLSVTN

SEQ ID NO: 14 - protein, 20 aa, Hevea brasiliensis
SSCGKCLSVTNTGTGAKTTV

SEQ ID NO: 15 - protein, 20 aa, Hevea brasiliensis
TNTGTGAKTTVRIVDQCSNG

SEQ ID NO: 16 - protein, 20 aa, Hevea brasiliensis
TVRIVDQCSNGGLDLDVNVF

SEQ ID NO: 17 - protein, 20 aa, Hevea brasiliensis
NGGLDLDVNVFRQLDTDGKG

SEQ ID NO: 18 - protein, 20 aa, Hevea brasiliensis
VFRQLDTDGKGYERGHITVN

SEQ ID NO: 19 - protein, 20 aa, Hevea brasiliensis
KGYERGHITVNYQFVDCGDS

SEQ ID NO: 20 - protein, 20 aa, Hevea brasiliensis
VNYQFVDCGDSFNPLFSVMK

SEQ ID NO: 21 - protein, 20 aa, Hevea brasiliensis
VDCGDSFNPLFSVMKSSVIN

SEQ ID NO: 22 - DNA, 28 bp, Artificial Sequence
  oligonucleotide primer
gcggaattcgagcaatgtggtcggcaag

SEQ ID NO: 23 - DNA, 38 bp, Artificial Sequence
  oligonucleotide primer
gcggtcgacttaattaattactgatgatttcataacgg

SEQ ID NO: 24 - DNA, 37 bp, Artificial Sequence
  oligonucleotide primer
gaattcgagcaagcaggtcggcaagcaggtggcaagc

SEQ ID NO: 25 - DNA, 37 bp, Artificial Sequence
  oligonucleotide primer
gcttgccacctgcttgccgacctgcttgctcgaattc

SEQ ID NO: 26 - protein, 43 aa, Hevea brasiliensis
EQCGRQAGGKLCPNNLCCSQWGWCGSTDEYCSPDHNCQSNCKD

SEQ ID NO: 27 - protein, 43 aa, Hevea brasiliensis
EQAGRQAGGKLCPNNLCCSQWGWCGSTDEYCSPDHNCQSNCKD

SEQ ID NO: 28 - protein, 43 aa, Hevea brasiliensis
EQAGRQAGGKLAPNNLCCSQWGWCGSTDEYCSPDHNCQSNCKD

SEQ ID NO: 29 - protein, 43 aa, Hevea brasiliensis
EQAGRQAGGKLAPNNLACSQWGWCGSTDEYCSPDHNCQSNCKD

SEQ ID NO: 30 - protein, 43 aa, Hevea brasiliensis
EQAGRQAGGKLAPNNLACSQWGWCGSTDEYCSPDHNCQSNAKD

SEQ ID NO: 31 - protein, 43 aa, Hevea brasiliensis; identical to SEQ ID NO: 26